(19) United States
(12) Patent Application Publication
(10) Pub. No.: US 2023/0395260 A1
(43) Pub. Date: Dec. 7, 2023

(54) Automated Medication Management And Intervention

(71) Applicant: FeelBetter Ltd., Giv'atayim (IL)

(72) Inventors: Yoram Hordan, Herzeliya (IL); Liat Primor-Marom, Giv'atayim (IL)

(21) Appl. No.: 18/032,038

(22) PCT Filed: Oct. 14, 2021
(86) PCT No.: PCT/IL2021/051223

§ 371 Date: Apr. 14, 2023

(30) Foreign Application Priority Data
Oct. 15, 2020 (IL) 278092

(51) Int. Cl. G16H 50/30; G16H 20/10

(52) U.S. Cl. CPC G16H 50/30; G16H 20/10

(57) ABSTRACT

Some embodiments are directed to a computer implemented system and method for medication regimen management. The computer implemented system and method are designed for monitoring a patient's medication regimen and automatically identifying medication risks. The computer implemented system and method are further designed for automatically generating recommendations for changes (interventions) in the medication regimen, to improve the medication regimen and avoid or at least reduce potential harm to the patient or to suffer from medication-related complications, as well as reduce the likelihood of the patient to be hospitalized.

### CROSS REFERENCE TO RELATED APPLICATIONS

This application is a national phase filing under 35 C.F.R. § 371 of and claims priority to International Application No. PCT/IL2021/051223, filed on Oct. 14, 2021, which claims the priority benefit under 35 U.S.C. § 119 of Israeli Patent Application No. 278092 filing date Oct. 15, 2020, the contents of which are hereby incorporated in its entirety by reference.

### BACKGROUND

The presently disclosed subject matter relates to a computer system and method for monitoring and identifying risks in a patient's medication regimen.

A medication associated risk or error is a failure in the treatment process of a patient and more specifically in the drug administration regimen assigned to a patient. Examples of medication risks and errors include, for example, erroneous prescription of drugs that may include the administration of an inappropriate drug and/or dosage, the administration of an ineffective drug, over prescribing, under prescribing, incorrect frequency or duration of drug administration, etc.

Medication associated risks and errors are very often harmful (or have the potential of being harmful) to patients and may directly induce adverse medical conditions or otherwise exacerbate existing medical conditions. Therefore, medication associated risks and errors have a general negative effect on a patient's health and increase morbidity rates. In addition, medication related risks and errors cause a significant increase in the hospitalization of patients and thus incur tremendous expenses for patients, as well as private and public health institutions.

### SUMMARY

While the task of identifying medication associated risks and errors in any medication regimen is challenging, due to the numerous interrelated factors that may have harmful effects on the patient, this task may become considerably more challenging in the case of complex patients such as those that exhibit comorbidity and polypharmacy. Comorbidity is a situation where a single individual suffers from multiple health problems (conditions), and, as a result, is under a complex medication regimen (polypharmacy) involving the use of multiple drugs. One non-limiting example may include an individual suffering from 3 chronic conditions while being administered with a medication regimen that includes 5 drugs or more. Consumption of multiple drugs has been known to affect the safety and effectiveness of the drugs, increase the frequency of drug-to-drug interaction, and, in general, pose a greater risk of causing harm to the patient.

The some embodiments of the presently disclosed subject matter includes a computer implemented system and method for medication regimen management. The disclosed system and method are designed for monitoring a patients medication regimen and automatically identifying medication risks. The disclosed system and method are further designed for automatically generating recommendations for changes (interventions) in the medication regimen, to improve the medication regimen and avoid or at least reduce potential harm to the patient or to suffer from medication-related complications, as well as reduce the likelihood of the patient to be hospitalized. Recommendations can be provided to, and applied for example by, health personnel, such as a physician or pharmacist. The disclosed methods can be applied on many patients concurrently thus providing a high-throughput tool.

According to some embodiments of the presently disclosed subject matter, personal health data of a patient that includes clinical and pharmaceutical data, obtained from electronic data resources, is analyzed for the purpose of generating a personalized risks map (or “risks map”), mapping various potential health risks that may be a result of the patient's medications or clinical situation.

Some embodiments of the presently disclosed subject matter further contemplates a graphical user interface executable by a computerized device (e.g. patients end device) which enables interaction with the patient for the purpose of obtaining from the patient additional (complementary) information not available in the patients electronic records. Some embodiments of the presently disclosed subject matter further contemplates a computerized device configured to run such interactive user interface.

Some embodiments of the presently disclosed subject matter further includes a processing circuitry configured to automatically generate and manage an interactive and dynamic smart questionnaire comprising of a set of questions, limited in number according to the attention span of the patient, and specifically adapted according to the personalized risks map of each specific patient for the purpose of obtaining in real-time, information with respect to possible health risks and experienced symptoms, selected according to their parameters including for example risk and severity.

According to a first aspect of some embodiments of the presently disclosed subject matter there is provided a computerized method of automatic identification of health risks in a medication regimen administered to a patient, the method comprising using a processing circuitry for:

obtaining a personalized risks map of the patient, the personalized risks map comprising a collection of health items, each health item is a data object corresponding to a respective medical condition identified as relevant to a health status of the patient; the personalized risks map includes medical data, wherein each health item comprises or is otherwise associated with specific medical data related to the respective medical condition;

identifying from among the collection of health items, based on the medical data and personal medical data of the patient, one or more risk related health items giving rise to a second collection of health items, wherein each risk related health item corresponds to a respective medical condition that is related to a potential health risk and/or error in the medication regimen of the patient;

determining a respective validity score for the one or more risk related health items, wherein the validity score indicates a level of certainty that the related potential health risk and/or error exists; and

generating, for at least one risk related health item having a validity score above a certain threshold value, at least one recommendation for an intervention dedicated for reducing the potential health risk and/or error.

In addition to the above features, the computerized method according to this aspect of the presently disclosed subject matter can optionally comprise one or more of features (i) to (xiii) below, in any technically possible combination or permutation:
i. Wherein the method further comprising generating the personalized risk map comprising:
   obtaining a medical database, the medical database comprising a plurality of health items (each health item in the medical database being a data object corresponding to a respective medical condition and comprises or is otherwise associated with clinical and/or pharmacological data related to the respective medical condition);
   comparing between the personal medical data of the patient and medical data available from the plurality of health items in the medical database; identifying health items which are relevant to the health status of the patient from among the plurality of health items in the medical database, according to the comparing; and adding the identified health items to the collection of health items in personalized risks map.
ii. Wherein the information in the personal medical data of the patient includes data about one or more of: one or more reported medical conditions that the patient has; one or more treatments that are being administered to the patient; and one or more medical measurements results that were administered to the patient.
iii. Wherein identifying health items which are relevant to the health status of the patient, further comprises:
   identifying in the personal medical data of the patient, data on a drug being administered to the patient;
   comparing the data with corresponding data related to the drug in the medical database, and identifying at least one potential health risk related to administration of the drug to the patient; and
   adding to the personalized risks map, at least one risk related health item that corresponds to a health condition related the health risk.
iv. wherein identifying health items which are relevant to the health status of the patient, further comprises:
   identifying in the personal medical data of the patient, data on a drug being administered to the patient; comparing the data with corresponding data related to the drug in the medical database, and identifying at least one potential health risk related to administration of the drug to the patient; and adding to the collection of health items, a risk related health item that corresponds to a health condition related the health risk. Where in some examples the drug is administered to the patient for treating a first medical condition and the risk related health item corresponds to a second medical condition.
v. wherein identifying health items which are relevant to the health status of the patient, further comprises:
   identifying in the personal medical data of the patient, data on a drug being administered to the patient; comparing the data with corresponding data related to the drug in the medical database, and identifying a potential health risk related to administration of the drug to the patient; identifying in the personalized risks map an existing health item corresponding to a medical condition related the potential health risk, updating the health item with data related to the drug; and increasing the validity score of the existing health item.
vi. wherein identifying health items which are relevant to the health status of the patient, further comprises:
   identifying in the personal medical data of the patient, a reported medical condition; adding to the personalized risks map a health item that corresponds to the reported medical condition. Where in some examples, identifying one or more risk related health items further comprises:
   processing the personal medical data and searching for a respective reported treatment that is being administered for treating the reported medical condition;
   in case a respective reported treatment is not found, or in case an error is found in the respective reported treatment, indicating the health item that corresponds to the reported medical condition as a risk related health item.
vii. Wherein the at least one potential health risk related to administration of the drug to the patient includes any one of: adverse drug reaction caused by the drug; and an adverse effect caused by drug-to-drug interaction of the drug with another drug being administered to the patient.
viii. wherein the determining the respective validity score of the one or more risk related health items comprises: identifying, in the personal medical data of the patient, parameters that support the certainty that the related potential health risk and/or error exists; and determining the respective validity score according to one or more of: number of parameters; type of parameters; and values of parameters. Wherein the parameters include for example, at least one medical measurement
ix. wherein the second collection of health items includes a subgroup of one or more risk related health items characterized by a validity score which is lower than a predefined threshold value;
   wherein the computerized method further comprises executing an interactive validation process dedicated for obtaining complementary data from the patient with respect to one or more health items in the subgroup, the interactive validation process includes providing a plurality of questions to the patient;
   the interactive validation process further comprising (A):
   prioritizing the health items in the subgroup and selecting a current health item having a highest priority;
   providing a current question to the patient dedicated for obtaining complementary data directly from the patient for the validation (i.e. increasing validation score above the threshold) of the current health item.
   responsive to the complementary data received from the patient, updating the personalized risks map comprising:
   updating the personal medical data of the patient to include the complementary data giving rise to updated personal medical data
   comparing between the updated personal medical data of the patient and medical data available from the plurality of health items in the medical database;
   based on the comparison, generating an updated second collection of one or more risk related health items and updating the validity score of one or more of the health items in the second collection;
   generating an updated subgroup of one or more risk related health items characterized by a respective validity score lower than the predefined threshold value; repeating (A) until a stop criterion is met.
x. Wherein the stopping criterion is any one of: the plurality of question are completed (were presented to the patient); and all health items in second collection have a validity score above the predefined threshold.
xi. wherein the interactive validation process, further comprises, prior to (A) providing at least one preliminary question to patient; and updating the personalized risks map.
xii. The computerized method further comprising:
   classifying the health items in the subgroup to a respective health category of a plurality of health categories, thereby providing a plurality of subsets of health items, each subset comprising one or more health items assigned to a certain health category;
   wherein (A) further comprising:
      prioritizing the health items in the subgroup and the categories and selecting a current health item or category having a highest priority;
      in case a health item is selected, providing the current question to the patient; and in case a category is selected, providing a survey question to the patient, the survey question is dedicated for obtaining complementary data from the patient for the validation respective subset of health items assigned to the category.
xiii. Wherein updating the personalized risk map further comprising:
   identifying an updated collection of health items which are relevant to the health status of the patient from among the plurality of health items in the medical database, according to the comparison, which constitute an updated personalized risks map; and the generating of the updated second collection further comprises:
   identifying from among the updated collection of health item, based on the medical data and the updated personal medical data of the patient, one or more risk related health items giving rise to the second collection of health items.

According to a second aspect of the presently disclosed subject matter there is provided computerized method of automatic identification of health risks in a medication regimen of a patient; the method comprising using a processing circuitry for executing an interactive validation process dedicated for validating health items in a personalized risk map; the personalized risks map comprising a collection of health items, each health item is a data object corresponding to a respective medical condition identified as relevant to a health status of the patient; wherein the collection of health items includes a plurality of risk related health items, wherein each risk related health item corresponds to a respective medical condition that is related to a potential health risk and/or error in the medication regimen of the patient; wherein the plurality of risk related health items include a subgroup of one or more risk related health items characterized by a validity score which is lower than a predefined threshold value;

wherein the computerized method further comprises executing an interactive validation process dedicated for obtaining complementary data from the patient with respect to one or more health items in the subgroup, the interactive validation process includes providing a plurality of questions to the patient;

wherein the computerized method further comprises executing an interactive validation process dedicated for obtaining complementary data from the patient with respect to one or more health items in the subgroup, the interactive validation process includes providing a plurality of questions to the patient;

the interactive validation process further comprising (A):

prioritizing the health items in the subgroup and selecting a current health item having a highest priority; providing a current question to the patient dedicated for obtaining complementary data directly from the patient for the validation of the current health item;

responsive to the complementary data received from the patient, updating the personalized risks map comprising:

updating the personal medical data of the patient to include the complementary data giving rise to updated personal medical data; comparing between the updated personal medical data of the patient and medical data available from the plurality of health items in the medical database; based on the comparison, generating an updated second collection of one or more risk related health items and updating the validity score of one or more of the health items in the second collection; generating an updated subgroup of one or more risk related health items characterized by a respective validity score lower than the predefined threshold value;

repeating (A) until a stop criterion is met.

According to a third aspect of the presently disclosed subject matter there is provided a computerized system for automatic identification of health risks in a medication regimen of a patient, comprising a processing circuitry configured to execute operations as described with reference to the second aspect above.

According to a fourth aspect there is provided a computerized system for automatic identification of health risks in a medication regimen administered to a patient, the computer system comprising a processing circuitry configured to:

obtain a personalized risks mop of the patient, the personalized risks mop comprising a collection of health items, each health item is a data object corresponding to a respective medical condition identified as relevant to a health status of the patient; the personalized risks map includes medical data, wherein each health item comprises or is otherwise associated with specific medical data related to the respective medical condition; identify from among the collection of health item, based on the medical data and personal medical data of the patient, one or more risk related health items giving rise to a second collection of health items, wherein each risk related health item corresponds to a respective medical condition that is related to a potential health risk and/or error in the medication regimen of the patient;

determine a respective validity score for the one or more risk related health items, wherein the validity score indicates a level of certainty that the related potential health risk and/or error exists; and generate, for at least one risk related health item having a validity score above a certain threshold value, at least one recommendation for a intervention dedicated for reducing the potential health risk and/or error.

The methods and system according to various aspects, can optionally further comprise one or more of features (i) to (xiii) listed above, mutatis mutandis, in any technically possible combination or permutation.

The presently disclosed subject matter further contemplates a non-transitory computer readable storage medium tangibly embodying a program of instructions that, when executed by a computer, cause the computer to perform a method as described above with reference to any one of the first aspect the second aspect and may optionally further comprise one or more of features (i) to (xiii) listed above, mutatis mutandis, in any technically possible combination or permutation

### BRIEF DESCRIPTION OF THE DRAWINGS

In order to understand the presently disclosed subject matter and to see how it may be carried out in practice, the subject matter will now be described, by way of non-limiting examples only, with reference to the accompanying drawings, in which:

FIG. 1 is high level schematic illustration of a system, according to some examples of the presently disclosed subject matter,

FIG. 2 is a block diagram schematically illustrating a more detailed view of the system, according to some examples of the presently disclosed subject matter,

FIG. 3 is a flowchart showing operations performed, according to some examples of the presently disclosed subject matter,

FIG. 4 is a flowchart showing high-level operations performed by the system, according to some examples of the presently disclosed subject matter,

FIG. 5 is a schematic illustration of a personalized risks map, according to some examples of the presently disclosed subject matter;

FIG. 6 is a flowchart showing operations performed during execution of a smart questionnaire, according to some examples of the presently disclosed subject matter;

FIG. 7 is a flowchart showing further operations performed during execution of a smart questionnaire, according to some examples of the presently disclosed subject matter;

FIG. 8 is another flowchart of operation performed during execution of a smart questionnaire, according to some examples of the presently disclosed subject matter; and

FIG. 9 is a flowchart showing the process of funneling of health-items and identifying candidate health-items for intervention, according to some examples of the presently disclosed subject matter.

### DETAILED DESCRIPTION OF EXEMPLARY EMBODIMENTS

In the drawings and descriptions set forth, identical reference numerals indicate those components that are common to different embodiments or configurations. Elements in the drawings are not necessarily drawn to scale.

Unless specifically stated otherwise, as apparent from the following discussions, it is appreciated that throughout the specification discussions utilizing terms such as “obtaining”. “determining”. “generating”. “comparing”. “adding” or the like, include an action and/or processes of a computer that manipulate and/or transform data into other data, the data represented as physical quantities, e.g. such as electronic quantities, and/or the data representing the physical objects.

The terms computer/computer device/computerized system, or the like, should be expansively construed to include any kind of hardware-based electronic device with a processing circuitry (e.g. digital signal processor (DSP), a GPU, a TPU, a field programmable gate army (FPGA), an application specific integrated circuit (ASIC), microcontroller, microprocessor etc.). The processing circuitry can comprise for example, one or more processors operatively connected to computer memory, loaded with executable instructions for executing operations as further described below.

The terms “client” and “server” as used herein below may include, but are not limited to, computers, server computers, personal computers, portable computers. Smartphones, appliances, watches, cars, televisions, voice-controlled assistants, tablet devices, or any other hardware computerized device configured with adequate processing and communication resources.

The operations in accordance with the teachings herein may be performed by a computer specially constructed for the desired purposes, or by a general purpose computer specially configured for the desired purpose by a computer program stored in a computer readable storage medium.

As used herein, the phrase “for example,” “such as”, “for instance” and variants thereof, describe non-limiting embodiments of the presently disclosed subject matter. Reference in the specification to “one case”, “some cases”, “other cases”, or variants thereof, means that a particular feature, structure or characteristic described in connection with the embodiment(s) is included in at least one embodiment of the presently disclosed subject matter. Thus, the appearance of the phrase “one case”, “some cases”, “other cases” or variants thereof does not necessarily refer to the same embodiment(s).

It is appreciated that certain features of the presently disclosed subject matter, which are, for clarity, described in the context of separate embodiments, may also be provided in combination in a single embodiment. Conversely, various features of the presently disclosed subject matter, which are, for brevity, described in the context of a single embodiment, may also be provided separately or in any suitable sub-combination.

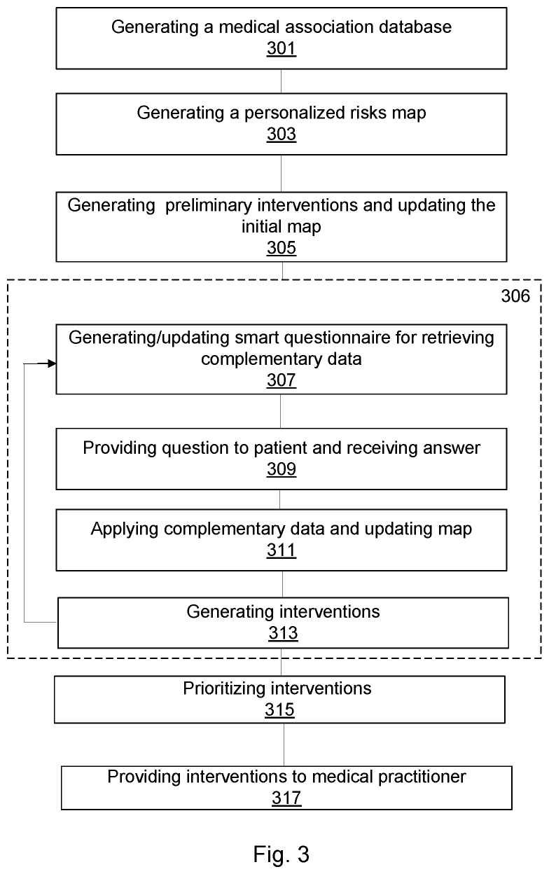

In embodiments of the presently disclosed subject matter, fewer, more and/or different stages than those shown in FIGS. 3,4,6,7 and 8 may be executed. In embodiments of the presently disclosed subject matter, one or more stages illustrated in FIGS. 3, 4, 6, 7 and 8 may be executed in a different order and/or one or more groups of stages may be executed simultaneously. For example, operations described with reference to blocks 415 and 417 may be executed simultaneously or in reverse order.

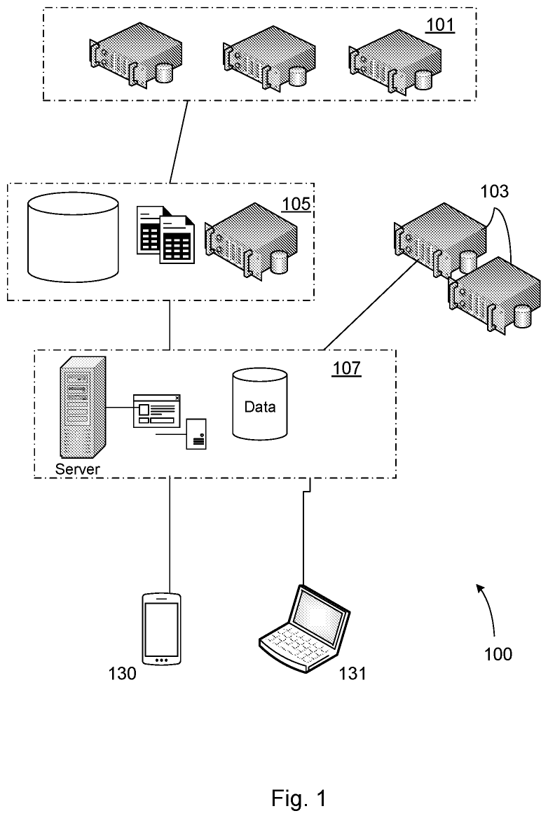

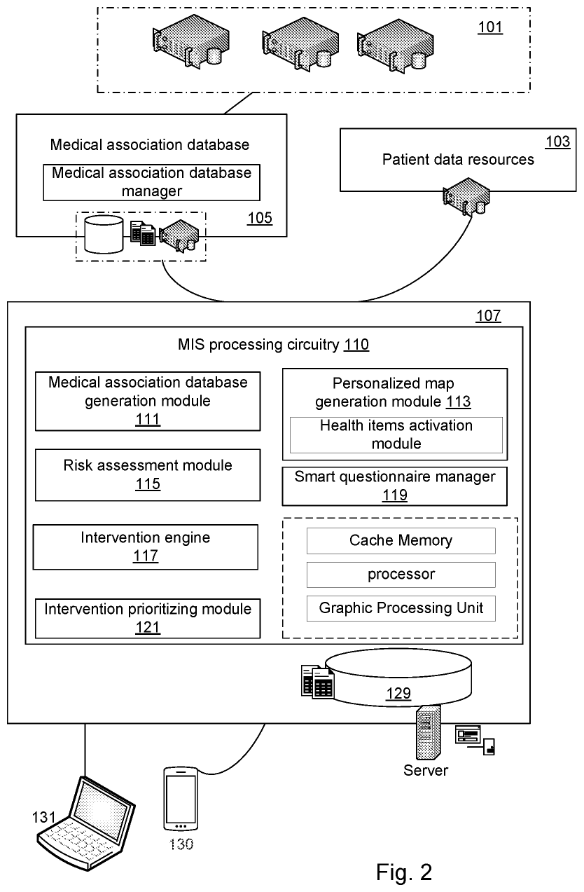

FIGS. 1 and 2 illustrate a general schematic of the system architecture in accordance with an embodiment of the presently disclosed subject matter. Elements in FIGS. 1 and 2 can be made up of a combination of software and hardware and/or firmware that performs the functions as defined and explained herein. Elements in FIGS. 1 and 2 may be centralized in one location or dispersed over more than one location. For example, MIS server 107 can be distributed over a plurality of computer devices, each possibly located at a different geographical location, or can be otherwise centralized in a single computer device. In other embodiments of the presently disclosed subject matter, the system may comprise fewer, more, and/or different elements than those shown in FIGS. 1 and 2. It should be understood that the specific division of the functionally of the disclosed system into specific parts as described below, is provided by way of example, and other alternatives are also construed within the scope of the presently disclosed subject matter.

Bearing the above in mind, attention is now drawn to FIG. 1 which shows a high-level view of a system, according to example of the presently disclosed subject matter. The network architecture in FIG. 1 is a general example which demonstrates some principles of the presently disclosed subject matter and may vary in structure and therefore should not be construed as limiting. System 16 may be implemented over any type of communication network. For example communication can be realized over any one of the following networks: the Internet, a local area network (LAN), wide area network (WAN), metropolitan area network (MAN), any type of telephone network (including for example PSTN with DSL technology) or mobile network (Including for example 3G, 4G or 5G mobile communication technologies), or any combination thereof.

System 166 is configured in general to provide a medical risk-stratification and intervention service (referred to herein in general as “medication intervention service” or “MIS” in short). As shown, a user (e.g. patient and/or medical practitioner) can use a client device (136/131) to communicate and interact with an MIS server 107 to obtain the medication intervention service as further explained below. Notably, multiple client devices can interact with the system simultaneously. Examples of specific functional elements of MIS server 107 are shown in FIG. 2 and are described below with reference to FIGS. 3-4 and 6-8. As shown in FIG. 2, according to some examples, MIS server device 107 comprises MIS processing circuitry 110 configured to execute several functional modules in accordance with computer-readable instructions implemented on a non-transitory computer-readable storage medium. Such functional modules are referred to hereinafter as comprised in the processing circuitry. In various examples, processing circuitry 110 comprises or is otherwise operatively connected to various components such as one or more computer processors and memory devices (e.g. cache memory).

According to some examples, MIS server 107 comprises or is otherwise connected over a communication network to a medical association database 105 (otherwise referred to as “medical database”), configured to store data lining between various medical conditions (otherwise referred to as “health conditions”) and related medical data. The related medical data includes clinical and pharmacological information such as:

medications and other treatments which are used for treating the respective medical condition, data indicating recommended usage or administration of the medication or treatment (e.g. dosage, frequency, duration, time of day, etc.), adverse drug reactions or symptoms that could be exhibited in patients that use these medications or treatment and other warnings related to drug usage such as mixture of different medications (DDI-drug to drug interaction) or warnings related to age groups or other affinity groups. Medical association database 105 can be configured to obtain the medical data from to one or more medication data resources 101 as further explained below.

In some examples, MIS server 107 is further connected to a patients' data resources 143 which can provide medical information on patients. Patients' data resources include for example electronic medical records (EMR) data-repositories (e.g. stored at various computer storage devices accessible by respective servers), that enable to obtain the EMR of a specific patient and/or claims data that relate to a specific patient.

Turning now to FIG. 3, this is a high-level flowchart of operations performed by system 166 (and more specifically by processing circuitry 110) according to some examples of the presently disclosed subject matter. During execution of a medical risk-stratification and intervention process, the medical status of a patient and the administered medical treatments (including administered drugs and other treatments, hereinafter “medication regimen”) currently being provided to the patient are analyzed in order to validate proper medication regimens, determine whether they include any medication errors and/or medication risks, and whether any optimization can be applied on the medical regimen in order to improve its efficiency in treating the relevant medical conditions and/or reduce potential risks to the patient.

In general, medication errors include any treatment in a medication regimen that fails to achieve its intended use and/or cause (or has the potential to cause) harm to the patient. In case a medication error is identified, recommendations for changes to the medication regimen (otherwise referred to herein as “interventions”), directed to remedy such errors and/or reduce related risks, are provided.

The medical risk-stratification and intervention process of a patient's medication regimen as disclosed herein can be performed as a recurring process, performed routinely, e.g. according to a specific scheduling scheme for the purpose of providing continuous evaluation and improvement to the patient's medication regimen. In other examples, the process can be executed in response to a specific incident or request. For example, following an acute medical event (e.g. a certain disease or injury). In yet another example, the process can be executed when a need arises to approve a change in the patient's medication regimen. e.g. a new drug is added to the patient's medication regimen.

For example, consider a patient submitting a request for renewal of a subscription to a certain drug. As part of the process of approving renewal of the subscription, a medical risk-stratification and intervention process can be executed, that automatically checks for any changes in the medical status of the patient that may have occurred since the last renewal, and whether these changes, if occurred, influence the risk related to the requested drug. Furthermore, as part of assessment of the patient's medication regimen, an analysis of the patient's trends over time and linkage to different clinical or pharmacological changes can be incorporated to better identify possible risks and causes and provide recommended interventions, accordingly.

Notably, some of the operations described herein below may not be executed every time. For example, generation of the medical association database as disclosed with reference to block 301 may not be repeated each time the process is executed. Rather, generation of the medical association database can be done once and then updated from time to time asynchronously from the execution of other operations related to the risk-stratification and intervention process.

According to some examples, at block 301 a medical association database is generated. The medical association database (otherwise referred to as “medication model”) is composed of a collection of individual health items. Each health item (or “health capsule”) is a data object that pertains to a specific medical condition or symptom that may inflict patients.

A non-exhaustive list of examples of medical conditions or symptoms that may be assigned with a respective health item include: hypertension (e.g. indicative of high blood pressure, over a certain threshold); hypotension, headaches; stomach aches; muscle aches; palsy; tremor, insomnia/sleep deprivation; weakness; nausea; vomiting; depression; anxiety; fatigue; confusion; constipation; diarrhea; allergic conjunctivitis; hypoglycemia; hyperglycemia hypokalemia; hypomagnesemia; hyponatremia, cytopenia, and so on.

Each health item comprises or is otherwise logically associated or linked to various data elements (e.g. data fields), where different data elements describe medical data related to the medical condition. Medical data in the medical association database 105 can include for example the following data elements: appropriate treatments and medication; various indication of the medical condition (such as blood test results or blood pressure results and how the are related to the medical condition) and/or possible related symptoms; other commonly associated medical conditions; warnings about medical conditions; pharmacologic data such as: recommended drug usage, such as dosing, time of day and frequency indexes; warning about drug usage including, adverse drug reactions (ADR), drug to drug interaction (DDI), other specific drug warnings, chemical components of drugs, etc.

As mentioned above, according to examples of the presently disclosed subject matter, the relevant medical data is obtained from medical data resources (101), which are generally available to medical staff and researchers and include for example: online medical literature, medical coding and classification systems (e.g. IDC codes of the World Health Organization and CPT codes are published by the American Medical Association), warning guidance databases, adverse drug reactions (ADR) databases, Drug to Drug interaction (DDI) information, drug duplications databases, medication warnings and guidance (e.g. NNT/NNH, impairments/grouping, Comorbidities), etc.

The medical resources 101 and possibly other data sources are analyzed to obtain the relevant medical data related to each medical condition that is added to the medical association database. This includes extraction of various causes, treatments (e.g. drugs) symptoms, indications, and risks, which are related to different heath conditions. In some examples, a deeper analysis is performed on the retrieved data. For example, pharmacological data can be analyzed for the purpose of determining the pharmacological/chemical components of drugs and the effect these specific chemical components may have regarding different medical conditions, risks and/or symptoms. One specific example is the analysis of the mechanism of action related to increased risk of falling caused by a specific drug and determining a specific drug component that may be the cause of falling.

A specific example of a health item and associated data is a sleep deprivation health item that can be associated with data elements related to one or more of: medical conditions that may be indicative of sleep deprivation, specific drugs that may cause sleep deprivation, combinations of two or more drugs (DDI) that may cause sleep deprivation, questionnaire based indexes that may point to sleep deprivation (e.g. Insomnia Severity Index), various medical measurements (e.g. blood pressure, blood test values) that are known to be associated with sleep deprivation, various treatments and drugs that may be administered for treating sleep deprivation, etc.

Another specific example of a health item and the associated data is a hemophilia health item that can be associated with data elements related to different factors that may cause/contribute/exacerbate hemophilia, different treatments that are administered for treating hemophilia and/or medical measurements/conditions that may be indicative of hemophilia. This includes drugs that may cause hemophilia, combinations of two or more drugs (DDI) that may cause hemophilia, other medical conditions and symptoms that are known to be associated with hemophilia, medical treatments that may cause or exacerbate hemophilia, various treatments and drugs that may be administered for treating hemophilia, and so on.

The medical association database 105 can further include specific information describing the details of each data element. For example, given a certain medical condition and a respective data element corresponding to a drug used for treating the medical condition, the specific details of the recommended drug administration are also provided as part of the database (e.g. various dosage indexes, according to age, weight, severity of symptoms, etc.). In another example, given a certain drug for treating a certain medical condition, which is associated with a certain warning or ADR, specific complementary conditions that may be required to render the warning or ADR effective or more severe, are also specified. For Instance, age (e.g. a warning relevant to a specific age group), measurements (a warning relevant if a medical measurement shows values above or below a certain value or within a certain range), specific (additional) medical condition (e.g. a warning is relevant if the patient is suffering from another specific medical condition), etc.

The abundance of health items recorded in the medical association database 105, which in some examples are defined in the hundreds or more, is intended to provide highly granular data describing specific medical conditions in detail, to thereby enable the representation and analysis of medical regimens of patients with high resolution and precision. It is noted however that the specific assignment of medical conditions to health items as well as the specific granulation of the health items may vary and the presently disclosed subject matter is not bound to any specific assignment or granulation.

In some examples the medical association database is generated and managed by a dedicated server. For example, server 105 can comprise a database manager implemented by a processing circuitry configured to parse the relevant data obtained from the various medical data sources 101 and identify within the data, medical data related to various medical conditions. Once the medical data is obtained, medical data related to a certain medical condition, is associated with (e.g. linked to) the respective health item that represents the medical condition. According to other examples, the medical association database is generated by MIS processing circuitry 110 (e.g. by medical association database generation module 111) configured for generating and managing the medical association database.

The medical association database 105 can be implemented using various techniques, data-structures and forms which are well known in the art. In one example, each entry in the medical association database (e.g. a row in a relational database) corresponds to a respective health item, representing in turn a specific medical condition (or symptom). The fields referenced by each entry can accommodate the medical data extracted from the data resources (101), including the different data elements related to the medical condition as explained above.

Notably, different data elements can be linked to more than one health item. For example, injected corticosteroids are known to have side effects that include the following medical conditions: stomach irritation, such as indigestion or heartburn; tachycardia (rapid heartbeat); nausea: insomnia and metallic taste in the mouth. Therefore, each respective health item representing any one of the above medical conditions, can comprise or otherwise be linked to a data element representing injected corticosteroids, indicating a possible causing agent (or “health item trigge”).

According to some examples, the medical association database 105 further includes information on possible health item triggers. The term “health item trigge” (or “trigge” in short) is used herein to include possible indications or causes of certain medical conditions. A trigger is linked in the medical association database to the respective health item representing the medical condition that may be potentially indicated or caused by the trigger.

One example is described above with respect to corticosteroids. Another example is Amitriptyline, which is a medicine used for treating various mental disorders. The list of ADRs related to Amitriptyline includes fatigue, xerostomia, headache, constipation, visual disturbance, dizziness, weight gain. The list of warnings related to Amitriptyline includes anticholinergic effect, cardiovascular event, conduction disorders, tachycardia, bone marrow suppression, bipolar disorder, and orthostatic hypertension. Thus, Amitriptyline can be considered as a trigger that may be related to the onset of any one of the above medical conditions. In some examples, each of these health items is associated with specific activation conditions, which must or should be complied to render Amitriptyline an effective trigger of the health item.

The triggers can be added to the medical association database 105 using various techniques, data-structures and forms which are well known in the art. Triggers can be implemented as data objects like health items. In some examples, the medical association database 105 can be designed with multiple references, such that each health item may reference one or more respective data elements and each data element may reference one or more health items. Specifically, data elements that represent triggers of medical conditions are linked to the respective medical condition which they trigger. According to other examples, the medical association database may include more than one individual data structure (e.g. data table), where at least one data structure links between the health items and the respective data elements, and at least one other data structure links between different data elements (health items triggers, causes and symptoms) and the respective health items which they trigger.

While all or most the health items constituting the medical association database 105 represents a full variety of medical conditions that may potentially inflict patients, each patient normally suffers from a subset of the medical conditions in the database.

At block 363 a personalized risks map is generated (e.g. by personalized map generation module 113 in MIS processing circuitry 110) for any specific patient. In general, a personalized risks map is composed of a collection (subset) of health items selected from the medical association database 105, which are relevant to the specific patient (referred to herein below as “relevant health item”) and provide the specific medical picture (status) of that patient, indicating the specific medical conditions and health risks that are relevant to the patient.

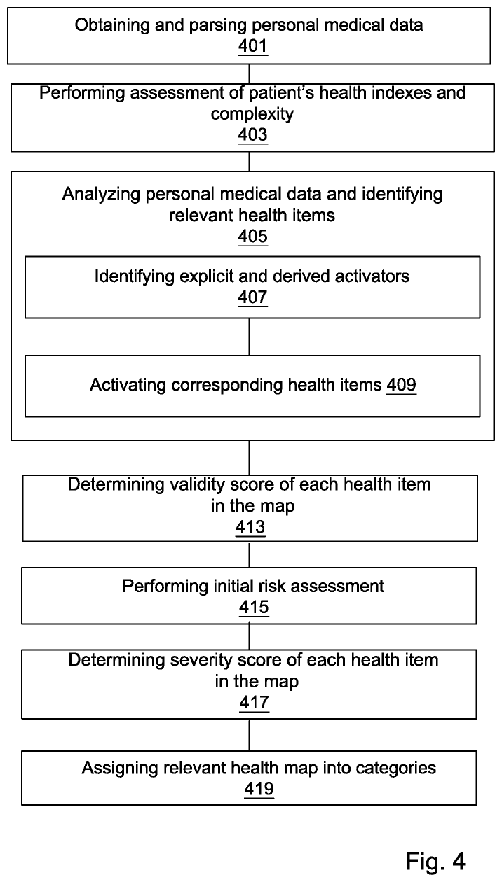

Turning now to FIG. 4, this illustrates is a flowchart of more detailed operations carried out during generation of a personalized risks map, according to some examples of the presently disclosed subject matter. The description reverts to FIG. 3 later below.

At block 401, available personal medical data (including for example, clinical and pharmacological data) of a patient is obtained and analyzed. In some examples, the personal medical data that is retrieved is stored in a dedicated data-repository operatively connected to system 107 (e.g., 129).

Patient' personal medical data includes for example; Patient's electronic medical records (EMR) and claims records including:
i. Personal information such as: patient's age, height, weight, gender, ethnicity and possibly other demographic data;
ii. Clinical information including, but not limited to:

Medical conditions that the patient is suffering from as well as medical background and history;

Patient's medical measurements, including for example, past blood tests and blood pressure results, urine tests, EEG, ECG, medical imaging results such as: X-ray, MRIL ultrasound. PET scan, CT, etc.;

Patients clinical indexes providing insights to the patient's clinical status (e.g. Patient Health Questionnaire-9 (PHQ9), Mini Nutritional Assessment (MNA), 3IQ, Creatinine Clearance Test (CCT), Child Pugh Score);

Information about the patient's frailty and ability to perform activities of daily living (e.g. geriatric assessment);

Various treatments administered to the patient other than drugs such as: dialysis, regular oxygen support, psychological treatment for psychiatric disorders, etc.
iii. Pharmacological information including:

Patient's medication regimen specifying the prescription drugs being administered to the patient, as well as the respective indications, dosages, frequencies, etc.; and

Consumed drugs and vitamin that are bought over the counter or supplements used and are recorded in the patient's EMR.

As further explained below, in addition to the personal patient's medical information mentioned above, subjective patient (complementary) data is also used by the processing circuitry 110 for generation of a personalized risks map of a certain patient. The subjective patient data is obtained by direct interaction with the patient in real-time using a user interaction platform running on a client device 130 (e.g. Smartphone). However, at the initial stages of the process, before interaction with the patient is performed and the subjective patient data is available, processing is done without the subjective data.

According to some examples, health indexes and the complexity of the patient are assessed (blocks 463). Health indexes may include renal impairment indexes, such as Creatinine Clearance Test, hepatic impairment indexes or other indexes associated with the patient's health. These indexes are used to identify specific risk related factors which are relevant to drug consumption under certain conditions such as renal impairment. To this end, the personal medical data of the patient (e.g. patient's EMR) is analyzed to determine whether it indicates a health index e.g. renal impairment index indicating that the patient is suffering from renal impairment.

As mentioned above, complex patients such as those that exhibit polypharmacy, suffer from multiple health problems, and are provided with a complex medication regimen involving the use of multiple drugs. In some examples, risk assessment of complex patients is different to that of other individuals with low complexity. For example, specific blood measurements associated with the patient's conditions may have different in-range definitions, which depend on the patient's complexity, and accordingly the dosages of medications that are appropriate for these patients may be different to those of the general population. Therefore, patient' complexity is evaluated, and the medical risk assessment of their medication regimen is adapted to restrictions related to their specific complexity. For example, a stricter medical risk assessment can be applied if the evaluated complexity is above a certain threshold.

In some examples, complexity of the patient is evaluated based on the number and type of medical conditions which are identified and/or treatments the patient is receiving regularly. For example, complexity increases with the increase in the number medical condition and/or the number of treatments (e.g., dialysis or regular usage of oxygen support).

The personal medical data of a patient is analyzed to identify relevant health items (block 465; e.g. by health items activation module in processing circuitry 110). In some examples, the identification of relevant health items is based on the identification of health item activators (block 407). The term “health item activato” is used hemin to include personal patient's medical data that may indicate that a certain health item in the medical association database 105 is relevant to a certain patient, thus “lighting-u” or “activatin” the health item and rendering it relevant to the patient's medical risk assessment.

As explained above, health items in the medical association database 105 are associated with one or more data elements corresponding to medical data including clinical and pharmacological information that is related to the respective medical condition. Accordingly, personal medical information of a patient, including that which is extracted from the patient's EMR, is crossed with medical data in the medical association database 105 (referred to herein also as “general medical data”) and health items found to be related to the personal medical data are activated (identified as relevant health items) and added to the collection of health items which constitute the patient's personalized risks map. In other words, the medical association databased indicates the connection between medical data and respective medical conditions. If the personal medical data of the patient is found to include data that is related to a certain medical condition, the respective health item representing the medical condition can be activated. Different medical conditions may be related to different health data, and therefore respective health items are activated by the specific health data to which it is related.

According to some examples, a first type of health item activators is “explicit health item activator”. This type of activators has a straightforward and explicit relationship with health items. An example of a direct activator is a medical condition reported in the patient's EMR (also referred to herein as “reported medical condition”) that indicates the relevance of a corresponding health item in the association database that represents the medical condition. For each medical condition disclosed in personal medical data of the patient, at least one health item representing the medical condition is activated—added to the patient's personalized risks map. For example, if the EMR indicates that the patient is suffering from migraines, the respective migraine health item is activated. Health items activated by direct activators are also referred to herein as “explicit health item” or “reported health item”.

As mentioned above, triggers include possible causes of certain medical conditions. Thus, triggers are a second type of activators (also referred to herein as “derived activator”) which are not directly related to a reported medical condition in the patient's personal health data, rather their effect on medical conditions is deduced by the system. Triggers are used for activating health items (herein below “derived health item”) which represent medical conditions which may possibly result from medication errors in the patient's medication regimen and accordingly derived health items mark potential medication errors and risks. As further explained below, triggers are also used for supporting the validity of explicit health items. As part of determining derived activators, treatments (e.g. drugs) which are being administered to the patient are retrieved from the patient's personal medical data and the ADRs and warnings relevant to these treatments (e.g. drugs) are extracted from the appropriate medical resources. Each ADR or warning represents a potential medical condition that a patient may be currently suffering from, or one that the patient may suffer from in the future.

To identify triggers and respective derived health items, computer logic is applied (e.g. by health items activation module in processing circuitry 110) on the medical data which is related to different health items in the map.

Several examples of computer logic operations carried out during the analysis of the health items are listed herein below:
a Assuming that according to a patient's EMR, the patient's is administered with a certain drug for treating medical condition A, the respective health item representing medical condition A is activated (explicit health item.) Assuming further that the medical association database 105 links drug A with one or more health items representing respective medical condition that may be induced as an adverse effect of the usage of drug A. These health items (derived health items) are activated as they represent health risks the patient is potentially facing, and the drug is identified as a potential trigger (cause) of these health items.
Assuming that according to a patient's EMR, the patient's is administered with two different drugs, drug A and drug B, one for treating medical condition A and the other for treating medical condition B, the respective health items representing medical condition A and medical condition B are activated (explicit health items). Assuming further that the medical association database 105 links these two drugs with one or more health items representing respective medical conditions that may be induced as a result of a drug-to-drug interaction between the drugs, when taken together. These health items are activated (derived health items) as they represent health risks the patient is potentially facing, and a combination of drugs causing the DDI is identified as a potential trigger of these health items.
Assuming that according to a patient's EMR, a certain measurement of a blood components in a patients' blood measurement (blood test) is out-of-range and that blood measurement is defined as a possible activator for a certain health item (representing a respective medical condition), that health item is activated, its related implications regarding specific drugs administered to the patient are identified, and the out-of-range measurement of the blood component are identified as a potential trigger of the health item.

The corresponding health items are activated according to the identified activators and triggers, for example activation may include marking the activated health items as relevant health items and adding the activated health items to the personalized risks-health map. The risks map can be implemented for example, as a dedicated data structure and/or database, where each entry represents a health item and stores information about the respective medical condition, respective activators and the personal medical data which is related to the activators (block 469).

Notably, the same health item may be activated by more than one medical condition. This may occur for example where the resolution of common clinical definitions is greater than the resolution of the definitions in the medical association database and accordingly a single health item may represent several related medical conditions. For example, health item representing hypotension may be activated by different classes of hypotension (orthostatic, postprandial neutrally mediated, severe, etc.).

Also, multiple medication related indications (e.g. ADRs or warnings) may activate the same health item. For example, the health item representing ‘Hypotension’ may be activated by a warning triggered by a medication containing an active pharmaceutical ingredient (API) Losartan (which may be administered to the patient for treating some other medical condition) and also may be activated by a different warning triggered by a medication containing an API Ramipril.

As is well known in the art and was demonstrated above with the example of Amitriptyline, a single drug may be related to many indications including various ADRs or warnings, while a small subset may be relevant to any specific patient. The long list of ADRs and warnings which is commonly disclosed in medication package inserts (also known as “patient information leaflet”) attached to medicines, is often ignored due to their generalization and the difficulty in identifying the ADRs and warnings which are indeed relevant to a specific patient. The system and methods disclosed herein aim to identify relevant ADRs and warnings as part of generation of the personalized risks map.

As part of the identification of triggers, potential risks identified in the personal medical data of a specific patient (including ADRs and warnings) are processed to identify triggers, and corresponding health items which are activated by the triggers and are therefore relevant to the specific patient. As the specifics of various risks, including ADR and warnings related to medications and other treatments, depend on many factors such as age, weight, gender, other drugs being used, complexity, renal impairment, medical history, and so on, such factors are taken into consideration during the analysis. As mentioned above, in some examples, the medical association database, may include specific data with respect to each of the health items and triggers indicating what are the conditions for their activation (referred to herein as “conditions of activatio”). The ADRs and warnings are screened according to the patient's personal data, and those ADRs and warnings which are not relevant to the specific patient (e.g., do not comply with the conditions of activation) are removed/ignored. For example, “drug related risks which are relevant to geriatric patients and not to younger individuals, are determined as irrelevant to patients of a younger age group. In another example, a warning related to a certain drug may activate a respective health item (rending it relevant) if another additional indication is reported in the patient's personalized risks map. For instance, a warning of constipation, associated with a drug reported in the patient's EMR, may activate the “constipation health ite”, if the EMR also indicates at least one of two symptoms. In case none of these two symptoms are reported the “constipatio” is not activated.

In some examples, a derived health item activated by a trigger, is considered relevant if them is at least one more activator that supports the trigger. For example, if there are at least two different derived activators that activate the same health item or if the same health item is activated by both a derived activator and an explicit activator it is considered relevant. In other examples, a derived health item is considered relevant if the patient's blood test indicates values within a certain range that support the indication of the trigger that activated the derived health item.

In some examples, overlaps between explicit health items and derived health items are identified and removed to maintain a single instance of each health item in the personalized risks-health map, thus in some cases an explicit health item and a derived health item are merged into a single health item corresponding to one medical condition.

Thus, the personalized risks map includes health items representing medical conditions which have some type of explicit record in the patient's personal medical data and the patient and/or the medical staff treating the patient may be aware of these conditions, as well as derived health items representing warnings about potential medical conditions which are not recorded in the patients' medical records and the patient and/or the medical staff may not be aware of their existence. Derived health items may also help to disclose asymptomatic medical conditions, that a patient may have, while being unaware of their existence. Furthermore, derived health items may also represent health risks which have not yet materialized, thus providing a predictive tool for identifying these risks and recommending interventions to reduce the likelihood of their materialization.

According to some examples, at block 413 during the process of generating a personalized risks map, a validity score is calculated (e.g. by risk assessment module 115) for each relevant health item. The validity score indicates the certainty that a health item identified as relevant to a certain patient is indeed relevant and that a health risk that is deduced from that health item indeed truly exists. In some examples the validity score is calculated based on a combination of parameters from different categories. These categories include clinical, pharmacological, and subjective parameters. Where clinical parameters include explicit activators (e.g. medical conditions explicitly reported in the patient's personal medical data and medical measurements and test results, such as, blood test, blood pressure, MRI, CT, etc.), pharmacological parameters include derived activators (e.g. drugs ADR or warnings determined based on the patient's drug regimen), and subjective parameters include answers provided by the patient to directed questions (e.g. answers to questions in the smart questionnaire), as further explained below.

In some examples, the validity score depends on the number of activators indicating the relevance of each health item, where the greater the number of activators, the higher the validity score. One example is that validity score of a derived health item which is activated by an ADR specified with respect to one drug that is administered to the patient and is further activated (or supported) by blood measurements, found in the patient EMR, that are known to be common to patients suffering from the medical condition represented by the derived health item, which may be greater than the validity score of a derived health item activated by ADR alone. A second example is the validity score of an explicit health item activated by a medical condition reported in the patient EMR and is further activated (or supported) by blood measurements, found in the patient EMR, that are known to be common in patients suffering from the medical condition represented by the derived health item, which may be greater than the validity score of a derived health item activated by the reported health item alone. A third example is the validity score of a derived health item which is activated by an ADR specified with respect to one drug that is administered to the patient and by a drug-to-drug interaction specified with respect two other drugs, which may be greater than the validity score of a derived health item activated by only the ADR or only the DDI.

The validity score may also depend on the type of activators. For example, activators from different categories (e.g. blood measurement, reported condition, health index) may have different contribution to validation (validation weight) as well. For each medical condition, the respective activators can be each given a respective validation weight, based for example, on the known relation between the activator and the respective medical condition. Thus, the system may include a validity scale, which indicates the validation weight of different activators for validating different medical conditions. In addition, the initial validity score of a health item, activated by a direct activator, is in some examples greater than the validity score of a derived health item, activated by a trigger (derived activator) as the validation weight of a direct activator is in general greater than that of a derived activator. In some examples, a derived health item, activated by a derived activator, may require one or more additional activators to confirm its validity.

The validity score may also depend on the specific value of an activator. For example, the correlation between certain blood measurement and certain a medical condition often depends on the specific value of the blood measurement.

The validity score assigned to each health item in the map can be stored for example in the entry representing the health item in the risks map.

In some examples, at block 415 initial medication error/risk assessment of explicit health items is carried out. During this stage, the relevant health items in the patient's personalized risks map are analyzed to determine medication errors/risks. To this end, computer logic is applied (e.g. by risk assessment module 115) on the medical data which is related to different health items in the map. As further explained below, according to one example, as part of the medical risk assessment, health items are classified as controlled or uncontrolled, where controlled health items include treated and balanced health items, and uncontrolled health items include untreated and/or unbalanced health items.

Several examples of computer logic operations carried out during the error/risk assessment of the health items are listed herein below:
a Activated health items in the personal risks map of the patient, are matched to information on administered drugs and other treatments obtained from the personal medical data of a patient, to determine whether all or most medical conditions are being treated. In case it is determined that a medical condition is properly treated with an appropriate drug or some other treatment, the respective health item is classified as “treated”. Otherwise, the respective health item is classified as “untreated”, where health items which are classified as untreated indicate a possible medication error/risk. As mentioned above, information regarding the drugs which are administered to a patient for treating specific medical conditions can be retrieved for example from the patient's personal medical data.

In some examples, a matrix can be generated for comparing all or most health items in the personalized risks map of a patient in an attempt to find a matching drug or other treatment for each medical condition represented by the health items. If a certain drug is known to be used for treatment of only one medical condition, and the patient has that condition, a match can be determined, and the respective health item is classified as treated. If a certain drug is used for treating two different medical conditions, and the patient has both, additional information obtained from the personal medical data of the patient can be used for determining which of the medical conditions is being treated using the drug. For example, if the EMR indicates a second drug that is used for treating one of the two medical conditions, it can be determined that the first drug is used for treating the remaining medical condition. Otherwise, complementary information may be needed for resolving the issue.
The dosages of each administered drug are compared to respective drug dosing ranges and thresholds, and in case it is determined that a dosage is out of range, e.g. exceeds a maximal allowed value, the respective health item is classified as “unbalanced” indicating a possible medication error or risk. Otherwise, if the dosage is within range, the health item is classified as “balanced”. Notably, in some examples, when determining proper drug dosing (including overdosing), the existence of renal failure is also considered, and the dosing thresholds are adapted to each specific patient according to renal impairment indexes.

Information on administered drugs and other treatments obtained from the personal medical data of a patient are further analyzed to determine whether the respective drug administration regimen is in accordance with the common medical recommendations provided by physicians. This includes, for example, dosage, frequency, delivery (e.g. oral or topical), etc. The specifics of the treatment are compared to the recommendations (stored for example in the respective health item), and in case a match is found, the respective health item is classified as “balanced”. Otherwise, the health item is classified as “unbalanced” indicating a possible medication error/risk.

The results of the error/risk assessment of each health item in the map, including the classification of each health item and the reason for the classification, can be stored in the entry representing the health item in the risks map data structure.

An example of a balanced health item is an activated high blood pressure health item, which includes: 1) a data element showing that the patient is being administered with a medicine for treating high blood pressure, where the drug administration details are according to the common recommendations; and 2) data elements showing information on recent blood pressure measurements values that are within the acceptable range.

Notably, as the specifics of various medications and other treatments depend on many factors such as age, weight, gender, other drugs being used, medical history, and so on, these factors are taken into consideration during the analysis (available for example in the medical association database 165). For example, correct dosing of administered drugs is evaluated according to age and weight/BMI based indexes. In another example, some drug related risks are relevant to geriatric patients and not to younger individuals. In case the patient is a complex patient and/or suffers from renal impairment, the recommended measurements and values are adapted to match the specific values recommended to such patients.

According to some examples, a severity score is determined for each of the relevant health items (block 417). The severity score indicates the level of risk or harmfulness (risk factor) of each health item to the patient. In some examples, the severity scores are calculated based on a collection of parameters. These parameters include for example: the body system(s) related to the health item (e.g., integumentary System, Skeletal System, Muscular System, Nervous System, Endocrine System, Cardiovascular System, Lymphatic System, Respiratory System, Digestive System, Urinary System), the organ(s) related to the health item (e.g., heart, lungs, limbs, liver, colon, etc.), the specific type of symptoms and the respective measured values and/or severity indexes (e.g., hypertension values, abnormal blood test values, insomnia severity index, etc.), specific personal data of the patient (e.g., age group, various indexes such as renal indexes) health item. In some examples, severity is determined according to various indexes categorizing medical conditions according to their risk to the patient. For example, severity of a high blood pressure condition may vary according to the specific blood pressure measurement values.

According to some examples, a severity score is calculated based on a compilation of these parameters. Different parameters can be given a respective severity weight based on a corresponding estimated risk related to the parameter and a combined severity score can be determined based on the compilation of these weights.

The results of the error/risk assessment of each health item in the mop, including the classification of each health item and the reason for the classification, can be stored in the entry representing the health item in the risks map.

According to some examples, several severity classes are defined, each class representing a different level of estimated impact on the patients' health, possibly resulting in complications and/or hospitalization. For instance, three main severity classes can be defined, where the first severity class represents low impact, the second severity class represents moderate impact, and the third severity class represents high impact. Controlled health items are assigned to a low risk class indicating little or no impact on possible deterioration in the patient's condition which may lead to complications or hospitalization. Uncontrolled (including unbalanced or untreated) as well as derived health items are assigned to either a moderate impact class or high impact class, depending on their estimated impact on the patient's condition.

Furthermore, in some examples, health items in the personalized risks map are divided into subsets, each subset representing a category of health items that share one or more common attribute (e.g. clinical/health related, body-system related, organ related) (block 419). In some examples, health items assigned to categories can be further assigned to sub-categories (e.g., according to the specific organs). A non-exhaustive list of examples of categories include:

Respiratory system; Digestive system; Skin; Movement; Mental and behavioral disorder, sleep disorder, and so on. By way of example, sleep disorder may include all or most health items related to sleep such as, insomnia, narcolepsy; and the mental and behavioral disorder category may include heath items such as: depression, anxiety, sleep deprivation, lack of appetite, etc.

As further explained below, division into categories contributes to improving the efficiency of the interaction process with the patient and optimization of the number and type of questions that are presented during this process.

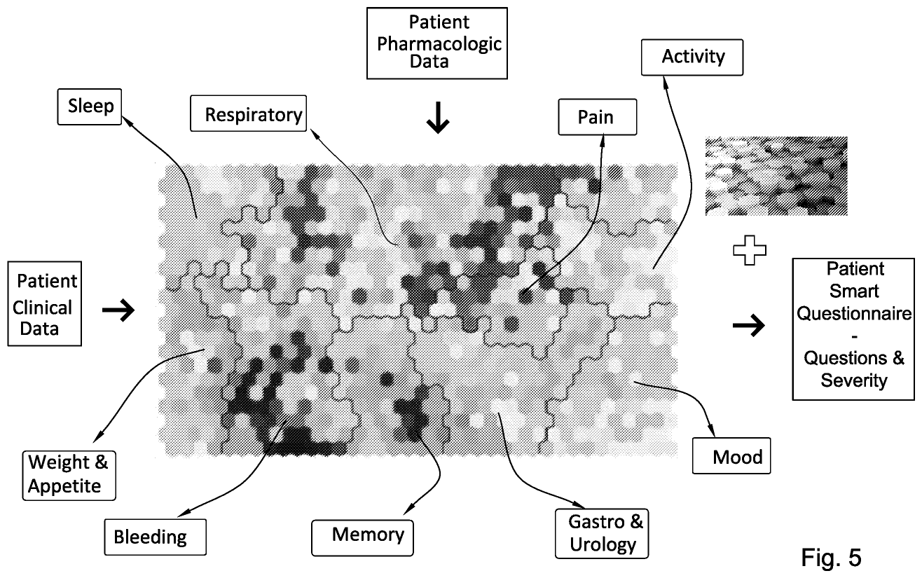

FIG. 5 is a schematic illustration showing a graphical representation of a personalized risks map, according to an example of the presently disclosed subject matter. As shown in the illustrated example, the relevant health items are graphically represented as hexagons, where the collection of hexagons provides a graphical representation of the personalized risks map.

The graphical representation of the map can further include information graphically displaying the classification (e.g., controlled, uncontrolled, treated, untreated), severity and validity of the health items. According to one example, the severity and validity score of a certain health item in the map can be displayed on the screen, e.g. responsive to a mouse hover over the health item. An example of a color-coding scheme of the different risk classes is illustrated in FIG. 5. In one example, green color is used to mark health items classified to a low severity class i.e. health items that represent medical conditions which have little or no potential of impacting the patients' health, such as balanced health items; red color is used to mark health items classified to a high severity class; and yellow color is used to mark health items classified to moderate severity class. Additional colors such as blue, light blue and yellow are used to mark various intermediate classes residing in between the three main classes mentioned above. FIG. 5 also shows the division of the relevant health items into different health categories. The graphical representation of the map can be displayed on a display device to be viewed by a user e.g. on end device 130 and/or 131.

As further explained below, the resulting personalized risks map, together with the severity and validity score, provides a valuable tool for identifying and screening possible medication errors and resulting health risks of a specific patient. The personalized risks map generated up to this point of the process is an initial form of the map (referred to herein as “initial map” otherwise referred to as “dry map”), which provides a partial picture of the medical condition of the patient as it combines clinical and pharmacological information obtained primarily from electronically available data sources but is missing complementary information.

For example, information not found in the clinical and pharmacological data sources includes: information on additional drugs which the patient is taking and are not listed in his EMR, possibly including over the counter (OTC) medications and supplements; updated measurement values such as home measurements of pulse, blood pressure values and sugar level values, which may be taken regularly at the patient's home; drug adherence information indicative as to whether the patient is actually taking the prescribed drugs listed in the EMR; updated weight; changes in habits including for example, commencement or cessation of smoking, drinking or physical activity; personal behavioral information which may not be available in the EMR such as depression, insomnia, anxiety, etc.

While the subjective medical data may have a critical effect on the evaluation of medication errors and possible risks in the patient medication regimen, it is not readily available and should be retrieved by directly interacting with the patient.

Reverting now to FIG. 3, in some examples, once the initial map is available, preliminary interventions are generated (block 365). To this end computer logic can be applied (e.g. by intervention engine 117) for the purpose of analyzing the previously identified medication errors or risks in the personalized-risks map (indicated as low, moderate and high severity health items), and suggesting possible interventions dedicated for remedying these errors.

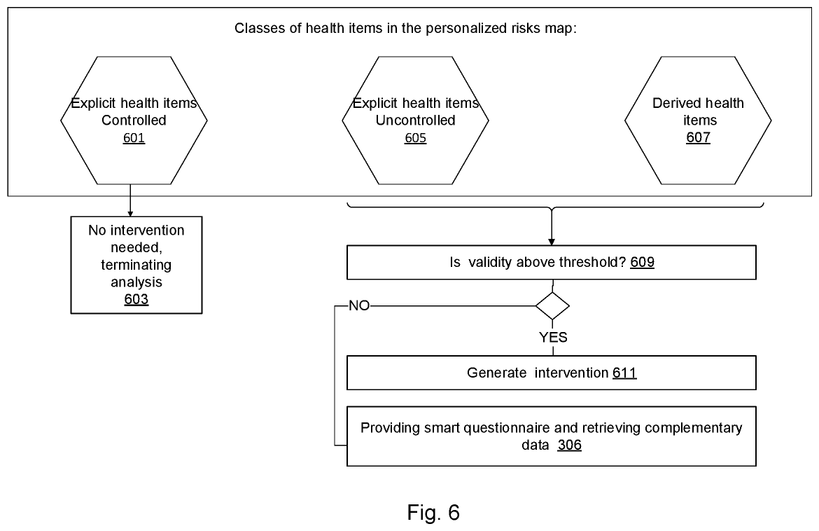

Turning to FIG. 6, it shows intervention decision logic, according to some examples of the presently disclosed subject matter (applied for example by intervention engine 117). Recall that health items in the initial map can be divided into a number of types, including: explicit health items which are controlled, explicit health items which are uncontrolled (including “untreated” and “unbalanced” health items) and derived health items. Controlled health items do not represent a significant risk to the patient and accordingly in general do not require any intervention. Interventions may be potentially applied on uncontrolled explicit health items and on derived health items (referred to collectively as “risk related health items”).

In case all health items in a personalized risks map of a certain patient are classified as controlled explicit health items and it is determined that no health risks have been identified. In such cases no intervention is required, and the analysis of the personalized risks map is terminated (block 603). In some examples, a message can be generated and provided e.g. to the client device, indicating that no interventions are needed.

In some examples, before the interaction with the patient for obtaining complementary data occurs, interventions are selectively generated to a subset of health items that includes uncontrolled explicit health items and derived health items which exhibit a validity score above a certain threshold value. These are health items that represent possible health risks to the patient with a high level of certainty, and therefore can be relied upon for generating a corresponding intervention. In case the initial map includes uncontrolled explicit health items or derived health items with a validity score sufficiently high (block 69), respective interventions an be generated for these health items (block 611, see details with respect to block 313 below).

A subgroup comprising other health items in the initial map, including uncontrolled explicit health items and derived health items, which have a validity score below the threshold (collectively referred to herein below as “open health items”) are further processed to increase their validity based on complementary data received directly from the patient. At block 306 an interactive validation process, that includes providing a smart questionnaire to the patient, is carried out. As mentioned above, an appropriate application that includes an interactive user interface can be executed on a computerized device operatively connected to MIS server 107 (e.g. client device 130 and/or 131) to enable interaction with the patient for the purpose of obtaining from the patient additional (complementary) information not available in the patients electronic records.

Examples of health items with a validity score above threshold that merits an intervention include explicit health items that are classified as untreated. This includes a scenario where an explicit health item in the map is activated based on information in the patients EMR indicating a certain medical condition, and the EMR does not indicate any drug that is being administered to the patient for treating the condition. Another example is an explicit health item in the map activated based on information in the patients EMR indicating a certain medical condition, and the type of drug indicated by the EMR as being administered for treating the condition in the prescribed dosage is incorrect (e.g. the dosage exceeds a maximal allowed dosage of the drug, or is below a minimal effective dosage of the drug). Another example includes a derived health item that indicates a certain risk (e.g. high blood pressure), which is also supported by one or more additional activators (e.g. blood pressure measurements that indicate high blood pressure). It is noted that the specific threshold of validity can vary and therefore the examples provided herein should not be construed as limiting.

In some cases, even if the validity of a health item is below threshold, a recommendation for intervention is provided to the user and the health item is not classified as an open health item. This scenario may occur for example, if a certain health item in the mop is activated based on information in the patients EMR indicating a certain medical condition or based on a trigger, and it is determined (e.g. by intervention engine 117) that a specific medical test is needed for validating the health item. Since a specific medical test is needed for the validation of the health item and in this case, questioning the patient will not help, a intervention recommending performing the test can be generated without further interaction with the patient.

In some examples, preliminary interventions generated during the preliminary stage can be stored (e.g. in data repository 129) until completion of the entire process and generation of all or most possible interventions. Examples of interventions are provided below with respect to block 313. As further explained below, according to some examples, health items identified as candidates for inducing the generation of respective interventions are added to a pool of candidate health items (e.g. stored in data repository 129) and once the entire process is complete and all or most candidate health items are identified (including those identified using complementary data obtained from the patient), a subset of health items is selected and interventions are generated for the health items in the subset.

Proceeding to block 307 in FIG. 3, according to the presently disclosed subject matter, an interactive and dynamic questionnaire (referred to herein as “smart questionnaire) is automatically generated and provided to the patient to obtain from the patient, during a certain interaction period, subjective information needed for increasing the validity of open health items in the personalized risks map and the respective medication errors or risks associated with these health items.

Users interacting with online content, and specifically with online questionnaires, normally have a limited attention time span, that, when spent, the user loses interest and stops the interaction. According to the presently disclosed subject matter, the smart questionnaire is generated as a set of prioritized questions which are limited in number according to the relevant attention time span of the patient. Given a certain attention time span, and the estimated interaction time required for each question, the number of questions that are incorporated in the smart questionnaire is calculated such that the interaction period with the patient is maintained within the limits of the attention time span. Thus, for a given time span, the questionnaire comprises a respective number of questions. By limiting the number of questions in the smart questionnaire and the respective interaction period required from the patient for answering the questions, the likelihood that the patient will answer all the questions is increased.

The attention time span can be defined with the same value for all patients or may vary from one patient to the next according to the patient's characteristics and the patient's specific attention time span. For example, different attention time span can be assigned according to age groups or other demographic data or combination thereof. In addition, or instead, attention time span can be assigned according to previous experience, where the time span assigned to a specific patient is based on the observed engagement of the patient with previous questionnaires. Thus, different patients may be assigned with a different questionnaire having a different number of questions determined according to their specific attention time span.

Since the number of questions that can be asked in a given questionnaire is limited, the personalized risks map is analyzed and the health items in the map are prioritized while taking into account their potential health risk to the patient. The prioritization of health items can be based on various parameters or combination thereof. The parameters can be obtained from each respective health item describing information about the medical condition in the context of a specific patient. According to one example, prioritization is done according to the respective severity score of health items, where a higher severity (indicating a greater health risk) is assigned with a higher priority. In some examples, prioritization of open health items and/or health categories is dependent (in addition or instead of severity) on the number of interventions induced by the health item or category. For example, a first open health item may be linked to a single ADR, while a second health item may be linked to 3 different ADRs and/or warnings (e.g., 3 different drugs in the medication regimens are ADRs of the same health item). Thus, by validating the second health item rather than the first, more health risks are addressed, as each ADR or warning may induce a respective intervention. When more than one parameter is used during prioritization, different parameters can be assigned with different weights, and the priority can be calculated as a weighted sum. Questions in the smart questionnaire are continuously updated in real-time, where the remaining questions that need to be presented to the patient during the interaction period are dynamically reassessed and updated in real-time as the questionnaire is taking place, based on the answers received from the patient to previous questions and their impact on the personalized risks map.

The computerized system and method disclosed herein enable to use the received answers to process and update the risks map, prioritize the remaining questions according to the updated risk map, and provide to the patient a consecutive question, dedicated for validating health items, in real-time without (or with little) delay. This approach is critical for maintaining the patient engaged with the questionnaire, while obtaining from the patient valuable information for determining accurate medications errors and risks. By prioritizing questions repeatedly, according to the current state of the risks map, it is ensured that the most severe medication risks are addressed first, thus reducing the risk to patients. Accurate detection and classification of medication errors or risks, which present high risk to the patient, enables to generate corresponding recommendations for intervention, which can help in turn, to reduce the likelihood of hospitalization of the patient and/or deterioration of his medical condition.

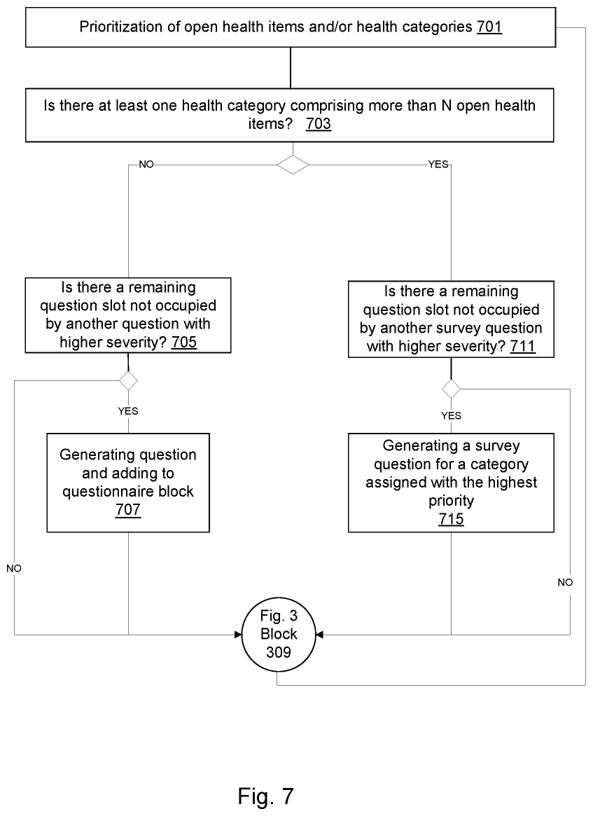

A more detailed review of examples of operations related to the generation and application of an interactive validation process using a smart questionnaire as mentioned with reference to block 307 in FIG. 3, is provided with reference to FIG. 7.

According to some examples, the interactive validation process may include providing to the patient preliminary questions before providing the smart questionnaire, at the onset of the interaction. These questions are directed for completing the patient's personal medical data and are not related specifically to any specific health item in the personalized risks map of the patient. The preliminary questions may include for example one or more of the following questions:
What is your weight?
Do you take any non-prescription drugs, vitamins or supplements?
Do you have an updated blood pressure value?
Do you have an updated blood sugar value?
Do you take all your prescribed drugs as prescribed?

For example, as part of the preliminary questionnaire, the user interface can display a list of all prescribed drugs as extracted from the patient's personal medical data and request the patient to mark those drugs which are actually being taken. In a further example, in case the patient indicates that he/she is not taking a certain prescribed drug, a follow-up question may be an enquiry to establish the reasons for not taking the drug, e.g. by providing a list of possible reasons, and asking the patient to mark the most relevant option.

The answers provided by the patient are added to the patient's personal medical data and are used for re-processing and updating the patient's personalized risks map. According to some examples, the respective severity scores and the validity scores of the health items are updated as well, according to the newly added data. During the update of the personalized risks map, the new data obtained by the preliminary questionnaire may induce changes to the map. In one example, health items previously not activated may be activated based on the new data retrieved by the preliminary questionnaire. In another example, an activated health item, previously classified as controlled, can be classified as uncontrolled, or vice versa. In a further example, the validity score of open health items may increase, where in case the validity exceeds the predefined threshold value, it is removed from the pool of open health items and may be provided with a respective recommendation for intervention.

For example, if the patient indicates that he/she regularly takes an over the counter (OTC) drug, which may cause an adverse drug-to-drug interaction (DDI) when taken along with another prescription drug, indicated in her EMR, a new trigger can be identified activating (or supporting) a corresponding health item. Assuming the health item is one that has already been activated by a direct activator, and the new trigger raises the validity score of the health item above a threshold, the validation score of the health item may rise above this threshold, and as a result the health item is removed from the pool of open health items.

Considering another example, where a patient reports body weight in the preliminary questionnaire. In some cases, body weight may influence the personalized risks map. For example, where the body weight changes the patients BMI value, the BMI may in turn influence various parameters used during generation of the personalized risks map. For instance, as mentioned above, drug administering indexes and protocols may depend on BMI values, thus a change in the patient's BMI may cause a health item, previously classified as controlled, to be classified as uncontrolled, or vice versa.

Considering a further example, where an ADR or warning related to a certain drug in the EMR of the patient triggers a derived health item, and a patient indicates that he/she does not consume the drug at all. In such cases the corresponding derived health item is deactivated (becomes irrelevant) and removed from the pool of open health items. At the same time, the classification of an explicit health item representing the medical condition that is treated by the drug can be changed from controlled (treated) to uncontrolled (untreated). The corresponding validity score is recalculated, and in case the validity score is below a threshold, the health item is added to the pool of open health items.

Changes in the open health items may also affect the number of open health items in different categories, and accordingly, further affect the generation of survey questions (directed to an entire category as further explained below) in the smart questionnaire. For example, assuming the validity of a health item is updated so it is removed from the pool of open health items, and, as a result, the number of health items in the corresponding category is reduced, this may cause a survey question which may have been otherwise generated, not to be generated.

When providing preliminary questions to the patient, the patient's attention time span and respective interaction period is taken into consideration. For example, the number of preliminary questions and the number of questions in the smart questionnaire is determined such that the time required for answering these questions does not exceed the allowed interaction period.

Operations described with respect to blocks 307-311 are related to generation and application of the smart questionnaire (executed for example by smart questionnaire manager 119). FIG. 7 is a flowchart showing operations carried out during generation and/updating of a smart questionnaire as mentioned in block 307 of FIG. 3, according to some examples of the presently disclosed subject matter. As mentioned above and demonstrated in FIG. 5, health items may be divided into categories, where different health items that share common features, are grouped into the same category.

According to the example described with reference to FIG. 7, application of the smart questionnaire further includes using survey questions, which are a special class of questions dedicated to enquiring about an entire category of health items. Survey questions with respect to the entire category can, in many cases, improve efficiency of the questioning process, since one question may replace the need to ask several individual questions with respect to different health items in the category. Notably, in some cases survey questions may not be applied. For example, in case the number of open health item is smaller than a certain number, survey questions may not be applied and in such case, operations described below with respect to categories we not executed.

At block 701 open health items in the personalized risks map and/or health categories, if such exist, are prioritized. As mentioned above, prioritization can be done based on various parameters or a combination thereof.

At block 703, it is determined whether the open health items in the personalized risks map comprises one or more subsets of at least N health items that belong to the same category, where ‘N’ represents a minimal number of health items that entails a survey question. In one exampleN>1; in another exampleN>2; and in yet another exampleN>3, etc.

In case none of the health categories comply with the required minimal number of health items, the process turns to the generation of questions for specific health items. Recall that the smart questionnaire allows a limited number of questions (herein “question slots”) according to the available interaction period with the patient. At block 705 it is determined whether them is a question slot in the questionnaire which is not occupied by a question that corresponds to a health item with priority that is higher than the top priority health item of the remaining health items in the subset of open health items (i.e. open heath items not assigned with a question in the questionnaire). Initially, the questionnaire is empty, and it is therefore occupied with questions pertaining to the open health items having the highest priority. Otherwise, if there is no available question slot in the questionnaire the process continues to block 309 in FIG. 3. Since updates made to the personalized risks map in response to answers provided by the patient to other questions in the questionnaire may result in the removal of questions from the questionnaire, it is possible that an open slot in the questionnaire will become available at a later time. If an open question slot is available, a question is generated for the current top priority health item and added to the questionnaire (block 707). In some examples, if more than one open slot is available, a question is generated for each available open slot, and thus, in the first iteration, a plurality of questions is generated for each question slot in the questionnaire.

Reverting to block 743 in case the personalized risks map incudes at least one category comprising a minimal number of open health items, the process turns to the generation of a survey question. In some examples, if more than one category complies with the requirement of a minimal number of open health items, the categories are prioritized e.g. according to their overall severity, where a higher severity is assigned with higher priority. According to some examples, calculation of the overall severity of a certain category is done based on the number of open health items in the category and severity of each health item, e.g. by calculating a weighted average. In another example, the priority is determined based on an aggregate score, such that a higher aggregate score in a certain category contributes to its assigned priority score.

In case it is determined that there is a remaining question slot in the questionnaire which is not occupied by another survey question with higher priority (block 711), at block 715 a survey question is generated for the category with the highest priority. Otherwise, if there is no available question slot in the questionnaire the process continues to block 369 in FIG. 3.

According to some examples, more than one question can be generated with respect to the same health item. According to further examples, different questions are associated with specific health categories, so that they can be combined under a survey question or a set of survey questions, when required.

The question with the highest priority can be displayed in graphical user interface of an application running on a computerized device 130 such as a Smartphone. In some examples, the question can have the format of a multiple-choice question, suggesting two or more possible answers to the patient. The patient interacts with the computerized device 130 and provides an answer to the question.

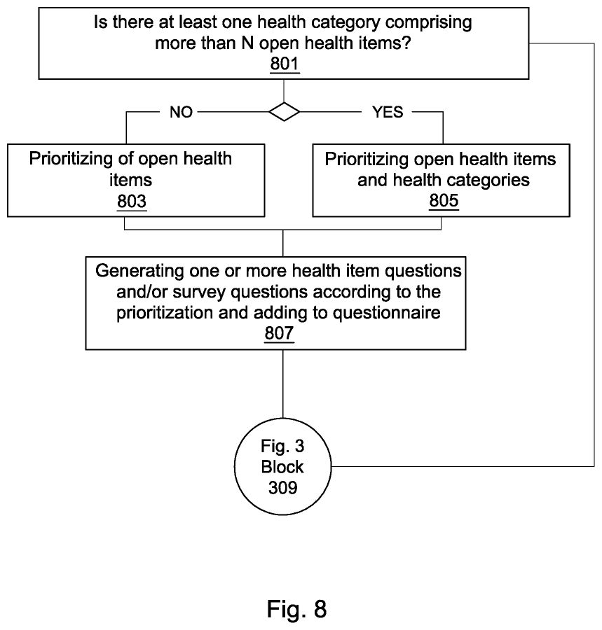

FIG. 8 is another example of operations flow carried out during generation and/updating of a smart questionnaire as mentioned in block 307 of FIG. 3, according to another example of the presently disclosed subject matter. According to this example, it is initially determined whether the open health items in the personalized risks map comprises one or more subsets of at least ‘N’ health items that belong to the same category (block 81), where, as mentioned above, ‘N’ represents a minimal number of health items that entails a survey question. In case none of the health categories comply with the required minimal number of health items, the process turns to the prioritization of individual health items (block 803). Otherwise in case the personalized risks map incudes at least one category comprising a minimal number of open health items, the open health items and health categories are prioritized (block 365). As explained above in general a category is assigned with a higher priority than individual open health items, however, in some cases the priority of a single health item may exceed the priority of a category. For example, in case a previous question that was presented to the patient, was a survey question related to a certain category and the answer from the patient indicated that respective category is relevant (e.g., patient answers that he is indeed suffering from heart problems), the individual health items in the respective category may be assigned with a higher priority than other categories, which have not been investigated thus far. This type of logic can be applied to ensure that once a category is identified as relevant, the following questions are directed for specific high priority health in the same category and thus provide a sequence of questions, which are maintained in the same context and thereby provide the patient with an improved user experience. To this end, system 107 (e.g., with the help of smart questionnaire manager 119) can be configured to apply a respective prioritization logic for prioritizing open health items and categories, which can be based on various parameters as explained above. Once the open health items and categories are prioritized a health item question or a survey question is generated for the current top priority health item or current top priority category and added to the questionnaire (block 307).

Reverting to FIG. 3, at block 309 the smart questionnaire is presented to the patient and an answer is received from the patient. The number of available questions in the questionnaire is decremented by 1. Since the questions in the questionnaire are directly derived from the health picture (status) of the patient as gleaned from the personalized risks map, changes in the personalized risks map may further induce changes to the smart questionnaire. Therefore, in some examples, each new question (a question corresponding to a new current highest priority health item) in the smart questionnaire is presented to the patient separately, and after the patient has provided an answer to the previous question, the risk map is updated based on the answer, and the smart questionnaire is re-processed and updated accordingly.

The data received from the patient is used for updating the patient personalized risks map (block 311). As explained above, the received answer is intended to increase the validity of one or more open health items. Furthermore, the patient's answer may affect the validity score of not only the specific health item or category, which induced the question, but also of other health items in the personalized risks map. The answer received from the patient is used for re-calculating the respective validation scores of the open health items, and in case the updated validation score of an open health item is above the predefined threshold, the respective health item is marked as validated, and is removed from the pool of open health items.

As mentioned above, the validation process, which includes generating and updating the questionnaire, occurs in real-time while the patient is interacting with the system (e.g. through a user application running on an end device) over a time that is limited by the interaction duration. As explained above with respect to the preliminary questionnaire, during this limited interaction duration, a dynamic process is carried out in the background, where, in response to an answer provided by the patient to a smart questionnaire question, the personalized risks map is updated. During update of the personalized risk map, the map generation is essentially repeated using the complementary information obtained from the patient via the questionnaire. The complementary data is used to enrich the patient's personal medical data, and the map generation is repeated (as described above with reference to block 363) using the enriched personal medical data.

Accordingly, personal medical information of a patient, including that which was previously extracted from the patient's EMR and the complementary data obtained directly from the patient, is crossed with medical data in the medical association database 105 (referred to herein also as “general medical data”) and relevant health items are identified and added to the collection of health items which constitute the patient's personalized risks map.

As part of the update process of the map, the validity scores of the relevant health items are updated, and an updated subgroup of open health items is determined accordingly (as described above with reference to FIG. 4; notably during update some of the operations described with respect to FIG. 4 may not be executed, e.g., block 401 and 417). As a result of the map update various changes can occur to the personalized risks map, including for example:
Health items in the medical association database, previously not classified as relevant can become classified as relevant and added to the risks map, while health items previously classified as relevant can become irrelevant and be removed from the risks map.
The validity of some health items may increase, while the validity of other health items may decrease. The change in the validity score may result in an updated subgroup of open health items, where some health items, may be added while other may be removed from the subgroup, according to the updated validity.
The priority of health items or categories may change and as a result the order of questions in the questionnaire may change as well.

Following the risks map update, the process returns to block 307 and the questionnaire is updated according to the now updated open health items and their respective validity and priority. A following question that pertains to a current highest priority health item or category that needs to be validated, is generated, and presented to the patient. This cycle as described with reference to blocks 307 to 311 is repeated for each question and answer in the smart questionnaire, until the interaction duration of the patient is terminated—which may occur once all questions have been asked or once all open health items have been validated. According to some examples, operation related to block 313 is also related to the generation and application of the smart questionnaire and is repeated in every cycle. As the identity of each upcoming question depends on the answer to the previous question, it cannot be determined until processing of the answer to the previous question is complete. The computerized system and method disclosed herein enable to obtain the complementary information from the patient for validating health items in real-time in a continuous manner.

The following are examples of questions that may be asked in the smart questionnaire and their possible implication on the patients personalized risks map.

If the patients EMR indicates that the patient is being treated within antidepressant that is associated with an ADR of dizziness and/or drowsiness, a respective question provided to the patient may enquire whether the patient experiences any of these symptoms or whether the patient has fallen or had trouble getting up. In another example, if the patients EMR indicates that the patient is being treated with a drug that is associated with an ADR of bleeding, a respective question provided to the patient may enquire whether the patient experiences any bleeding (e.g., nasal, rectal, urinary or gums). In case the answer received from the patient is positive, the validity of the respective health items is increased. Otherwise, if the answer is received from the patient is negative, the validity of the respective health items is decreased.

Similarly, if a derived health item has been activated by a DDI resulting from two drugs reported in the patents EMR, a question in the smart questionnaire may enquire whether the patient is indeed experiencing the symptoms related to the DDI. In case the answer received from the patient is positive, the validity of the respective health items is increased.

The examples below demonstrate how questions in the questionnaire can unpredictably change in real-time, due to complementary data received from the patient and the following risks map update.

Consider a derived health item that has been activated by an ADR or warning related to a certain drug in the EMR of the patient and which has been validated by blood test values within a certain range. A question in the smart questionnaire may enquire whether the patient is indeed experiencing the relevant symptom (adverse drug response) that is suspected of being triggered by taking the drug. In case the patient indicates that he/she does not experience the adverse drug reaction, or the symptoms related to the DDI, during the map update, the respective health item is deactivated and classified as irrelevant (with a validity score above threshold). As a result, the related questions are removed from the questionnaire and other questions take their place.

Likewise, in case certain health items are activated by certain drug induced ADRs, a question in the smart questionnaire may enquire whether the patient indeed takes these drugs. In case the patient indicates that he/she does not take these drugs, during the map update, the respective health items are deactivated and classified as irrelevant (with a validity score above threshold). As a result, the related questions are removed from the questionnaire and other questions take their place.

Assuming the patients EMR reports that the patient suffers from hypertension and is properly treated with an appropriate drug, a respective explicit health item is added to the map and classified as controlled. A question in the questionnaire may enquire about blood pressure measurements, which are regularly applied by the patient at home. In case the patient indicates that the blood pressure measurements (taken for example over the last month), are above a certain threshold, during the map update, the respective health items is classified as uncontrolled. As a result, new questions may be added to the questionnaire enquiring about the reasons for the uncontrolled health items, possibly replacing other questions assigned with lower priority.

Consider another example, where the personalized risks map includes health item A related to a certain medical condition, which is treated with drug A. Since drug A is known to be related to a certain ADR (e.g., headaches), a question in the questionnaire enquires whether the patient indeed suffers from the ADR. If the answer provided by the patient is positive, changes may be induced to the personalized risks map during a following map update.

A respective health item (health item B) related to constipation (medical condition A) is activated. As a result of the activation of health item B, a third health item is activated (health item C, which is related to a certain medical condition C, which is specified as a warning related to drug c, prescribed to the patient. The warning and respective health item C are characterized by a condition of activation, which limits activation of health item C by the warning only if the patient also suffers from at least one of, medical condition A (e.g., constipation) and medical condition D (e.g., heartburn), since the complementary data indicates that the patient indeed suffers from medical condition A, health item C is activated. To validate health item C a new question may be added to the questionnaire enquiring whether the patient suffers from heartburn, if the answer is positive the warning related to drug C can be validated. This example demonstrates again how an answer to a question related to one health item induces a new question related to a different health item in a manner that is unpredictable before the answer to the question is obtained.

In some examples, an answer to a question asked with respect to a first health item provides complementary data that may reduce the validity of a second health item, while the validity of the second health item is supported by other activators (e.g. blood test results). In such case, further complementary information may be needed for increasing the validity score of the second health item above threshold to render it valid. Thus, a new question may be generated and added to the questionnaire (assuming it is justified by the priority of the second health item) for the purpose of substantiating the validity of the second health item.

The above scenario can occur for example in case the second health item was assigned, before the complementary information has been received, with a validity score above the threshold and the decrease in its validity score caused by the answer provided for the first health item brought its validity score below threshold. The above scenario can also occur in case the second health item was assigned, before the complementary information has been received, with a validity score below threshold and the decrease in its validity score caused by the answer provided for the first health item brought the validity score further below threshold. Notably, in the first case, the second health item becomes an open health item only as a result of the complementary information, and therefore the answer to the question asked with respect to the first health item caused a question to be added to the questionnaire with respect to health item that otherwise would not have been subject to a question. In the second case, although the second health item was an open health item, a follow up question is added to the questionnaire to reconcile the contradicting information, i.e. the activator on one hand and the complementary data contradicting it on the other.

Another example is related to a survey question in the smart questionnaire dedicated for enquiring whether the patient suffers from any health problem in a certain health category. For example, this may be asking the patient whether he suffers from any type of pain. Assuming a patient's answer to a survey question refutes a suspicion of a medical condition in the health category (e.g. pain category), all open health items in the category are removed from the pool of risk related health items. Thus, in such cases, the remaining questions in the questionnaire will not be related to the category that was the subject of the survey question.

Assuming, alternatively, that a patient's answer to a survey question supports the validity of a suspected health category, and by that supports the validity of all health items in the category, there may be several possible outcomes, including for example:
If the validity score of all health items in the category is updated to a value above a threshold to be considered validated, all health items in the category are removed from the subset of open health items, and a respective intervention may be generated for each health item in the category. Thus, in such cases, other questions in the questionnaire will not be related to this category.
If the validity score of only part of the health items in the category is updated to a value above a threshold to be considered validated, only those health items are removed from the subset of open health items, and a respective intervention may be generated for these health items. In such cases, other questions in the questionnaire may be generated for the purpose of validating the remaining health items in the health category.

Recall that the smart questionnaire is programmed to target and validate open health items characterized by high priority (e.g., high severity score). Thus, at the end of the validation process, health items with the highest priority are expected to be validated or to be discarded (“false positives”). According to some examples, an intervention is generated immediately after the validation of a health item as part of the cyclic flow of block 307 to 313, while in other examples, the interventions are generated only after completion of execution of the interactive validation process for all the validated health items. As mentioned above, in some examples, interventions generated as part of the validation process can be used for prioritizing the health items during the following cycle, e.g., based on the respective number of interventions generated for each health item.

At block 313 interventions are generated for the now validated health items. As explained above, computer logic can be applied for the purpose of analyzing errors or risks associated with the validated health items and for suggesting possible interventions dedicated for remedying these errors and alleviating the related risks (e.g. by intervention engine 117). More specifically, interventions are generated with respect to the drugs that are found to be the source of a health risk or error (which are represented by respective health items). As part of the generation of the recommendations for interventions, the information in the validated risk related health items is processed to determine which drug is related to the respect risks or errors and what is the nature of the risks or errors and to accordingly provide recommendations for changes in the patient's health regimen to reduce or eliminate the risk error.

Intervention engine 117 can be configured for the purpose of generating interventions recommendations to analyze the health items and extract from each health item data indicative of the detected error or risk and the suspected cause for the error or risk.

A non-exhaustive list of examples of possible recommendations for intervention is provided:

A first type of intervention is “replace drug”, recommending changing one or more drugs which are being administered to the patient. For example, in case a derived medical condition is triggered by an adverse drug reaction, changing the related drug may be recommended. In case a derived health item is triggered by a drug-to-drug interaction, changing one of the related drugs may be recommended.

A second type of intervention is “add drug” or “remove drug”, recommending adding a new drug or removing an existing drug from the currently administered medication regimen.

A third type of intervention is a recommendation to perform a certain test (e.g. blood test) or measurement to further validate an identified risk related to a drug.

A fourth type of intervention is a recommendation to reduce a dosage of a drug (e.g. due to overtreatment of a condition) or increase the dosage (e.g. due to lack of effectiveness).

A fifth type of intervention may be a recommendation to monitor the patient regarding a specific measurement or symptom that may be associated with an identified risk related to a drug.

In some cases, a recommendation for an intervention may not be conclusive. For example, assuming a health item is associated with three different drugs, where all three drugs have a similar adverse affect on the patient, it may be difficult to determine which one is the cause of a certain observed symptom. Thus, in some cases, the recommendation includes a few options, leaving it to the discretion of the medical practitioner to decide which intervention to follow.

An example of the processing applied on the personalized risk map is provided herein below:

Health Item A

One health item in the risks map is an explicit health item A representing medical condition A, which is being treated by administering drugs a1 and a2. As explained above this health item is activated based on information disclosed in the patient's personal medical data. The error/risk assessment processing indicates that health item A is controlled, i.e., drugs a1 and a2 are being properly administered for treating medical condition A.

Health Item B

A second health item in the risks map is a derived health item. Health item B was activated for representing a potential risk of medical condition B, which may be an ADR of the use of drug a1 (drug a1 a being a trigger of derived health item B). Health item B was further validated by blood tests results which support the possibility that the patient is suffering from medical condition B and therefore its validation score is above the threshold to be considered validated.

Health item C

A third health item in the risks map is an explicit health item C representing medical condition C, which is being treated by administering drugs c. The error/risk assessment indicates that health item c is uncontrolled. In this case the error/risk assessment processing found that the patient is suffering from renal impairment and while the dosages administered to the patient should be adapted according to the patient's renal impairment index, the patient is being administered with the dosage prescribed to individuals which are not renally impaired.

Health Item D

A fourth health item in the risks map is a derived health item D. Health item D was activated for representing a potential risk of medical condition D, which may result from the interaction between drug c and drug a2 (the drug-to-drug interaction between the drugs being the trigger of derived health item D). Health item D was not validated by other activator and is therefore classified as an open health item that requires further validation.

Complementary data is obtained from the patient during application of the questionnaire indicating that the patient is not suffering from medical condition D and is therefore classified as not relevant.

According to this example, recommendations for intervention may include:
A recommendation to replace drug a1 with an alternative drug which is not characterized by the s-me ADR. Notably, the drug has been replaced although the health item is classified as controlled.
A recommendation to adapt the dosage of drug c in compliance with drug dosing of the renal impairment index.

Once the smart questionnaire is terminated, there may be some health items with higher validity than others, and, accordingly, in some examples, interventions can be classified, e.g., one group comprising interventions related to health items with a validity score above a first threshold, and a second group comprising interventions related to health items with a validity score above a second threshold, lower than the first. Interventions in the first group are provided with an operational recommendation to be executed by the physician, and interventions in the second group are provided with a recommendation to further look into and/or monitor the related medical condition and/or potential risk.

Considering the fact that too many interventions may be overwhelming, confusing, and may cause the clinical practitioner to ignore the recommendations altogether, m some examples all or most the potential interventions that were generated are prioritized to thereby generate a prioritized list of interventions per medication (block 315). As mentioned above, interventions can be collected in a pool of interventions and stored (e.g. in data repository 129) until completion of the entire process and generation of all or most possible interventions. Once the process is complete, interventions in the pool are prioritized (e.g. by intervention prioritization module 121).

If, for example, there are 10 possible interventions that would alleviate the risks related to the respective health items (e.g. reduce the risk of hospitalization), MIS processing circuitry 110 can be configured to prioritize the interventions in a manner that strives to reduce the risk to the minimum possible. The output is the list of recommended interventions ordered according to their contribution in reducing the risks to the patient (e.g. being hospitalized). In some examples, only a subset of the candidate interventions in the pool are presented as items to be performed in a timely manner (e.g. S top interventions in the list), and the other interventions are displayed as secondary interventions to be performed at a later stage, or only after the patient's condition has stabilized.

An intervention priority score can be calculated for each intervention, enabling to prioritize the interventions. In one example, interventions are prioritized according to the following scheme:

In some examples, each intervention (provided with respect to a given drug) is assigned with a score for each one of the following criteria: safety, effectiveness, indication and adherence, where the priority of the criteria and the respective scores are as follows:
First (highest) priority: Safety—interventions that are dedicated to resolving medication issues that pose a risk to the safety of the patient and may cause medical harm if they go unaddressed.
Second priority: Effectiveness—interventions that are dedicated to resolving medication errors that result in ineffective medication but have a lower risk of causing harm to the patient in addition to the ineffective treatment of a respective medical condition.
Third priority: Indications—interventions that are dedicated to adding medications for treatment of a currently active condition of the patient that are not balanced, or removal of medications that are being consumed by the patient, but are not needed, based on the patient's current medical condition.
Fourth priority: Adherence—interventions that are dedicated to resolving medication risks that result from lack of adherence of the patient to the medication regimen. These interventions include suggesting an alternative drug or other treatment that the patient is more likely to adhere to (e.g. as an adverse drug response is less common with the alternative drug).

The score can be calculated based on the combination of these individual score parameters. Once finalized, the interventions can be provided to the client (block 317). As mentioned above, the client can be the patient himself and/or a clinical practitioner, such as a physician or a pharmacist. Information on the process, including a patient's personalized health risk map, questions and answers, recommended interventions, etc. can be displayed on a computerized device screen (136/131) which is operatively connected to system 107.

It will also be understood that the system according to the presently disclosed subject matter, may be a suitably programmed computer. Likewise, the presently disclosed subject matter contemplates a computer program being readable by a computer for executing the method of the presently disclosed subject matter. The presently disclosed subject matter further contemplates a machine-readable non-transitory memory tangibly embodying a program of instructions executable by the machine for executing the method of the presently disclosed subject matter.

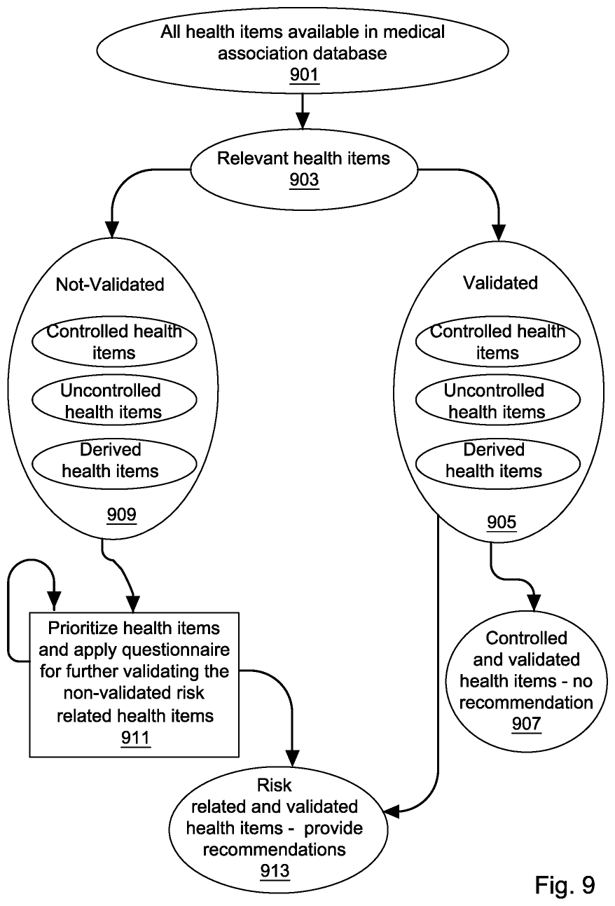

FIG. 9 is a flowchart showing a high-level view of the funneling of health items and identifying candidate health items for interventions, according to some examples of the presently disclosed subject matter. Initially, a subset of relevant health items is identified and added to the personalized risk map of specific patient (991-983). The subset of relevant health items is processed together with the personal medical data of the patient, and the health items in the subset are classified according to their validity, where the relevant health items can be divided into two subgroups, a first subgroup comprising health items with validity scores above a threshold, i.e. validated (965), and a second subgroup comprising health items with validity scores below a threshold, i.e. none validated (969). Each one of the two subgroups may include controlled explicit health items and risk related health items (including uncontrolled explicit health items and derived health items). Controlled health items which are validated may not require further recommendations (967).

Risk related health items in the first (validated) subgroup can be marked as health items that can be provided with recommendations (913). Risk related health items in the second group (non-validated, also referred to as “open health items”) undergo an interactive validation process executed for obtaining complementary data directly from the patient (911). The process includes generating and providing a questionnaire to the patient dedicated for validating health item in the second subgroup. Based on the complementary data obtained from the questionnaire the relevant health items which induced the question may become validated. The process reverts to block 983 where the risk map is updated using the newly obtained data potentially resulting in changes the validity of other relevant health items in the risks map, where none-validated health items may become validated and vice versa, thus affecting the health item which are part of the second subgroup. The process repeats until the interaction is over. Recommendation may then be provided to part or all of the health items which are classified as risk related and validated (block 913).

It is to be understood that the presently disclosed subject matter is not limited in its application to the details set forth in the description contained herein or illustrated in the drawings. The presently disclosed subject matter is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the present presently disclosed subject matter.

1-34. (canceled)

35. A non-transitory computer-readable storage medium comprising instructions that when executed by a computer, cause the computer to carry out a method for automatic identification of health risks in a medication regimen administered to a patient, the method comprising:
generating a personalized risks map of the patient, the personalized risks map comprising a collection of health items, each health item is a data object corresponding to a respective medical condition identified as relevant to a health status of the patient and comprises medical data related to the medical condition;
the collection of health items comprising a second collection of one or more risk related health items, wherein each risk related health item is identified based on personal medical data of the patient and corresponds to a respective medical condition related to a potential health risk and/or error in the medication regimen of the patient;
determining a respective validity score for the one or more risk related health items, wherein the validity score indicates a level of certainty that the related potential health risk and/or error exists;
wherein determining a validity score comprises executing a real-time computer-interactive validation process over a limited time period of user interaction dedicated for obtaining complementary data from the patient with respect a subgroup of risk related health items characterized by a validity score lower than a certain threshold;
the real-time computer-interactive validation process comprising (A):
prioritizing the health items in the subgroup and selecting a current health item having a highest priority;
providing a current question to the patient dedicated for obtaining complementary data from the patient;
responsive to the complementary data received from the patient, updating the personalized risks map comprising:
updating the personal medical data of the patient to include the complementary data giving rise to updated personal medical data;
updating the personalized risk map and generating an updated second collection of one or more risk related health items based on the updated personal medical data;
updating a validity score of one or more of the health items in the second collection;
generating an updated subgroup of one or more risk related health items characterized by a respective validity score lower than the threshold value;
repeating (A) until a stop criterion is met; and
generating, for at least one risk related health item at least one recommendation for an intervention dedicated for reducing the potential health risk and/or error.

36. The non-transitory computer-readable storage medium of claim 35, wherein the method further comprising:
classifying the health items in the subgroup to a respective health category of a plurality of health categories, thereby providing a plurality of subsets of health items, each subset comprising one or more health items assigned to a certain health category;
wherein (A) further comprising:
if the subgroup comprises at least one subset with more than N health items:
prioritizing the health items in the subgroup and the categories and selecting a current health item or category having a highest priority;
in case a health item is selected, providing the current question to the patient;
in case a category is selected:
providing a survey question to the patient, the survey question is dedicated for obtaining complementary data from the patient corresponding to a respective subset of health items assigned to the category; and
updating a validity score of the health items in the subset.

37. The non-transitory computer-readable storage medium of claim 35, wherein generating the personalized risk map comprises:
obtaining a medical database, the medical database comprising a plurality of health items;
comparing between the personal medical data of the patient and medical data available from the plurality of health items in the medical database;
identifying health items which are relevant to the health status of the patient from among the plurality of health items in the medical database, according to the comparison; and
adding the identified health items to the collection of health items in personalized risks map.

38. The non-transitory computer-readable storage medium of claim 37, wherein the method comprising for identifying a risk related health item:
identifying in the personal medical data of the patient, data on a drug being administered to the patient;
comparing the data with corresponding data related to the drug in the medical database, and identifying at least one potential health risk related to administration of the drug to the patient; and
adding to the personalized risks map, at least one risk related health item that corresponds to a medical condition related the health risk.

39. The non-transitory computer-readable storage medium of claim 37, wherein the method comprising for identifying a risk related health item:
identifying in the personal medical data of the patient, data on a drug being administered to the patient;
comparing the data with corresponding data related to the drug in the medical database, and identifying a potential health risk related to administration of the drug to the patient;
identifying in the personalized risks map an existing health item corresponding to a medical condition related the potential health risk, updating the health item with data related to the drug; and
increasing the validity score of the existing health item.

40. The non-transitory computer-readable storage medium of claim 35, wherein the method comprising for identifying a risk related health item:
comprising for identifying health items which are relevant to the health status of the patient:
identifying in the personal medical data of the patient, a reported medical condition;
adding to the personalized risks map a health item that corresponds to the reported medical condition.

41. The non-transitory computer-readable storage medium of claim 40, wherein the method comprising for identifying a risk related health item:
processing the personal medical data and searching for a respective reported treatment that is being administered for treating the reported medical condition;
in case a respective reported treatment is not found, or in case an error is found in the respective reported treatment, indicating the health item that corresponds to the reported medical condition as a risk related health item.

42. The non-transitory computer-readable storage medium of claim 41, wherein the error includes an unbalanced treatment caused by any one of: incorrect drug; incorrect drug dosage; incorrect delivery frequency; and incorrect delivery method.

43. The non-transitory computer-readable storage medium of claim 38, wherein the at least one potential health risk related to administration of the drug to the patient includes any one of: adverse drug reaction caused by the drug; and an adverse effect caused by drug-to-drug interaction of the drug with another drug being administered to the patient.

44. The non-transitory computer-readable storage medium of claim 35, wherein determining the respective validity score of the one or more risk related health items further comprises: identifying, in the personal medical data of the patient, parameters that support the certainty that the related potential health risk and/or error exists; and calculating the respective validity score according to one or more of: number of parameters; type of parameters; and values of parameters.

45. The non-transitory computer-readable storage medium of claim 44, wherein the parameters include at least one medical measurement.

46. The non-transitory computer-readable storage medium of claim 37, wherein updating the personalized risk map comprising:
identifying an updated collection of health items which are relevant to the health status of the patient from among the plurality of health items in the medical database, which constitute an updated personalized risks map.

47. The non-transitory computer-readable storage medium of claim 35, wherein the method comprising: displaying the current question in a user interface executed on a computer device while the user is interacting with the computer device.

48. The non-transitory computer-readable storage medium of claim 35, wherein the method comprising providing the one or more recommended interventions to a medical practitioner.

49. The non-transitory computer-readable storage medium of claim 35, wherein the at least one recommendation for an intervention includes a plurality of recommendations, each dedicated for reducing a respective potential health risk and/or error, the method further comprising providing data indicative of prioritizing of the plurality of recommendations. A computerized system for automatic identification of health risks in a medication regimen administered to a patient, the computer system comprising a processing circuitry configured to:
generate a personalized risks map of the patient, the personalized risks map comprising a collection of health items, each health item is a data object corresponding to a respective medical condition identified as relevant to a health status of the patient and comprises medical data related to the medical condition;
the collection of health items comprising a second collection of one or more risk related health items, wherein each risk related health item is identified based on personal medical data of the patient and corresponds to a respective medical condition related to a potential health risk and/or error in the medication regimen of the patient;
determine a respective validity score for the one or more risk related health items, wherein the validity score indicates a level of certainty that the related potential health risk and/or error exists;
wherein the processing circuitry is configured for determining a respective validity score for the one or more risk related health items to execute computer-interactive validation process over a limited time period of user interaction dedicated for obtaining complementary data from the patient with respect a subgroup of risk related health items characterized by a validity score lower than a certain threshold;
the real-time computer-interactive validation process comprising (A):
prioritizing the health items in the subgroup and selecting a current health item having a highest priority;
providing a current question to the patient dedicated for obtaining complementary data from the patient;
responsive to the complementary data received from the patient, updating the personalized risks map comprising:
updating the personal medical data of the patient to include the complementary data giving rise to updated personal medical data;
updating the personalized risk map and generating an updated second collection of one or more risk related health items based on the updated personal medical data;
updating a validity score of one or more of the health items in the second collection;
generating an updated subgroup of one or more risk related health items characterized by a respective validity score lower than the threshold value;
repeating (A) until a stop criterion is met; and
the processing circuitry is configured to generate, for at least one risk related health item at least one recommendation for an intervention dedicated for reducing the potential health risk and/or error.

51. The computerized system of claim 50, wherein the processing circuitry is further configured to:
classify the health items in the subgroup to a respective health category of a plurality of health categories, thereby providing a plurality of subsets of health items, each subset comprising one or more health items assigned to a certain health category;
wherein (A) further comprising:
if the subgroup comprises at least one subset with more than N health items:
prioritizing the health items in the subgroup and the categories and selecting a current health item or category having a highest priority;
in case a health item is selected, providing the current question to the patient;
in case a category is selected:
providing a survey question to the patient, the survey question is dedicated for obtaining complementary data from the patient corresponding to a respective subset of health items assigned to the category; and
updating a validity score of the health items in the subset.

52. The computerized system of claim 50, wherein the processing circuitry is configured for generating the personalized risk map to:
obtain a medical database, the medical database comprising a plurality of health items;
compare between the personal medical data of the patient and medical data available from the plurality of health items in the medical database;
identify health items which are relevant to the health status of the patient from among the plurality of health items in the medical database, according to the comparison; and
add the identified health items to the collection of health items in personalized risks map.

53. The computerized system of claim 52, wherein the processing circuitry is configured for identifying a risk related health item to:
identify in the personal medical data of the patient, data on a drug being administered to the patient;
compare the data with corresponding data related to the drug in the medical database, and identifying at least one potential health risk related to administration of the drug to the patient; and
add to the personalized risks map, at least one risk related health item that corresponds to a medical condition related the health risk.

54. The computerized system of claim 52, wherein the processing circuitry is configured for identifying a risk related health item to:
identify in the personal medical data of the patient, data on a drug being administered to the patient;
compare the data with corresponding data related to the drug in the medical database, and identifying a potential health risk related to administration of the drug to the patient;
identify in the personalized risks map an existing health item corresponding to a medical condition related the potential health risk, updating the health item with data related to the drug; and
increase the validity score of the existing health item.

55. The computerized system of claim 50, wherein the processing circuitry is configured for identifying health items which are relevant to the health status of the patient to:
identify in the personal medical data of the patient, a reported medical condition;
add to the personalized risks map a health item that corresponds to the reported medical condition.

56. The computerized system of claim 55, wherein the processing circuitry is configured for identifying a risk related health item:
processing the personal medical data and searching for a respective reported treatment that is being administered for treating the reported medical condition;
in case a respective reported treatment is not found, or in case an error is found in the respective reported treatment, indicating the health item that corresponds to the reported medical condition as a risk related health item.

57. The computerized system of claim 56, wherein the error includes an unbalanced treatment caused by any one of: incorrect drug; incorrect drug dosage; incorrect delivery frequency; and incorrect delivery method.

58. The computerized system of claim 53, wherein the at least one potential health risk related to administration of the drug to the patient includes any one of: adverse drug reaction caused by the drug; and an adverse effect caused by drug-to-drug interaction of the drug with another drug being administered to the patient.

59. The computerized system of claim 50, wherein the processing circuitry is configured for determining the respective validity score of the one or more risk related health items to: identify in the personal medical data of the patient, parameters that support the certainty that the related potential health risk and/or error exists; and calculate the respective validity score according to one or more of: number of parameters; type of parameters; and values of parameters.

60. The computerized system of claim 52, wherein the processing circuitry is configured for updating the personalized risk map to:
identify an updated collection of health items which are relevant to the health status of the patient from among the plurality of health items in the medical database, which constitute an updated personalized risks map.

61. A computerized method for automatic identification of health risks in a medication regimen administered to a patient, the method comprising using a processing circuitry for:
generating a personalized risks map of the patient, the personalized risks map comprising a collection of health items, each health item is a data object corresponding to a respective medical condition identified as relevant to a health status of the patient and comprises medical data related to the medical condition;
the collection of health items comprising a second collection of one or more risk related health items, wherein each risk related health item is identified based on personal medical data of the patient and corresponds to a respective medical condition related to a potential health risk and/or error in the medication regimen of the patient;
determining a respective validity score for the one or more risk related health items, wherein the validity score indicates a level of certainty that the related potential health risk and/or error exists;
wherein determining a validity score comprises executing a real-time computer-interactive validation process over a limited time period of user interaction dedicated for obtaining complementary data from the patient with respect a subgroup of risk related health items characterized by a validity score lower than a certain threshold;
the real-time interactive validation process comprising (A):
prioritizing the health items in the subgroup and selecting a current health item having a highest priority;
providing a current question to the patient dedicated for obtaining complementary data from the patient;
responsive to the complementary data received from the patient, updating the personalized risks map comprising:
updating the personal medical data of the patient to include the complementary data giving rise to updated personal medical data;
updating the personalized risk map and generating an updated second collection of one or more risk related health items based on the updated personal medical data;
updating a validity score of one or more of the health items in the second collection;
generating an updated subgroup of one or more risk related health items characterized by a respective validity score lower than the threshold value;
repeating (A) until a stop criterion is met;
and
generating, for at least one risk related health item at least one recommendation for an intervention dedicated for reducing the potential health risk and/or error.
